(19) United States
(12) Patent Application Publication
(10) Pub. No.: US 2002/0119452 A1
(43) Pub. Date: Aug. 29, 2002

(54) Osteoarthritis tissue derived nucleic acids, polypeptides, vectors, and cells

(76) Inventors: Deborah J. Phippard, Chesterfield, MO (US); Geetha Vasanthakamur, St. Louis, MO (US); Stanton Dotson, Chesterfield, MO (US); Xiao-Jun Ma, San Diego, CA (US)

Correspondence Address: Rachel Polster, Patent Department Central, Monsanto/G.D. Searle, P.O. Box 5110, Chicago, IL, 60680-5110, US

(21) Appl. No.: 09/765,231

(22) Filed: Jan. 18, 2001

Related U.S. Application Data
(60) Provisional application No. 60/176,523, filed on Jan. 18, 2000.

(51) Int. Cl. A01K 67/00; C12Q 1/68; G06F 19/00; G01N 33/48; G01N 33/50; C07H 21/04; C12N 9/00

(52) U.S. Cl. 435/6; 800/8; 435/325; 435/320.1; 702/20; 435/183; 536/23.2

(57) ABSTRACT

This invention relates to nucleic acids derived from osteoarthritis (OA) tissue as well as compounds and compositions made using these nucleic acids. A variety of nucleic acid and polypeptide compounds and compositions are described and specifically disclosed. The nucleic acids or polypeptides may be contained within vectors or host cells and then used to produce agents such as nucleic acids, polypeptides, fragments of polypeptides, antibodies, and variants of each. These molecules can be used to diagnose or treat osteoarthritis, or to analyze the disease-modifying activity of OA drugs. Cells containing one or more nucleic acids or polypeptides of the invention can also be used as targets in high-throughput screening methods, particularly in screening for compounds designed to identify compositions affecting osteoarthritis.

The present application claims priority under Title 35, United States Code, §119 of U.S. Provisional application Serial No. 60/176,523 filed Jan. 01, 2000.

### FIELD OF THE INVENTION

This invention relates to nucleic acids derived from osteoarthritis (OA) tissue as well as compounds and compositions made using these nucleic acids. A variety of nucleic acid and polypeptide compounds and compositions are described and specifically disclosed. The nucleic acids or polypeptides may be contained within vectors or host cells and then used to produce agents such as nucleic acids, polypeptides, fragments of polypeptides, antibodies, and variants of each. These molecules can be used to diagnose or treat osteoarthritis, or to analyze the disease-modifying activity of OA drugs. Cells containing one or more nucleic acids or polypeptides of the invention can also be used as targets in high-throughput screening methods, particularly in screening for compounds designed to identify compositions affecting osteoarthritis.

### BACKGROUND OF THE INVENTION

Osteoarthritis (OA) is a slowly progressing degenerative disease characterized by cartilage destruction that affects one or more joints. It is the most prevalent articular disease, and can severely impair mobility and lower extremity function (Ling and Bathon, J Am Geriatr Soc 46:216-25 (1998)). The term osteoarthritis implies an inflammatory disease. Although inflammatory cells may be present, OA is considered to be an intrinsic disease of cartilage in which biochemical and metabolic alterations result in its breakdown. Diagnosis is typically based upon radiological examination as well as clinical observations such as localized tenderness and bony or soft tissue swelling, joint function, severity of pain and the ease with which everyday functions such as climbing stairs can be performed. Characteristic radiographic findings include subchondral bone sclerosis, subchondral cysts and osteophytosis. Although joint space narrowing is considered to be a marker for articular cartilage thinning, in patients with early OA, who do not have radiographic evidence of bony changes, joint space narrowing alone does not accurately indicate the status of the articular cartilage. Similarly osteophytosis alone, in the absence of other radiographic features of OA, may be due to aging rather than OA. The correlation between the pathological severity of OA and symptoms is poor. Many individuals with radiographic changes of advanced OA have no symptoms. The risk factors for pain and disability in subjects with OA is still poorly understood.

Estimating OA disease progression presents more of a challenge. The current “gold standard” measure of disease progression is the change in joint space caused by articular cartilage loss observed using plain X-rays (Mazzuca et al., Osteoarthritis and Cartilage 5:217-226 (1997)). Since changes are small (1-2 mm per year), a minimum of one year is required before sufficient changes have occurred to be detectable. An additional problem with this technique is the requirement that the joint must be in exactly the same position for each radiological examination.

Current osteoarthritis therapies treat the associated pain and inflammation, but do little, if anything, to modify the course of the disease. Drug trials of potential disease modifying osteoarthritis drugs (DMOADs) require a minimum of one year before drug efficacy can be assessed.

Therefore there is a great need for biological markers and design assay systems that will predict disease modifying efficacy of Osteoarthritis drugs (DMOADS) within a significantly shorter time frame than the current year required for assessment of radiological changes. Also, disease markers that will detect early disease and allow early intervention with pharmacologic agents are much needed, but they are presently unavailable (Ling and Bathon, J Am Geriatr Soc 46:216-25 (1998)).

### SUMMARY OF THE INVENTION

Genetic and Bioinformatic Analyses

New opportunities for markers of OA disease and potentially for novel disease modifying therapeutics are described in this invention. These sequences have been identified using detailed genomics and bioinformatics analyses combined with our understanding of the pathology of OA. This invention identifies nucleic acid sequences and genes involved in the biology of OA and represents targets from which diagnostic tools, such as monoclonal antibodies, can be generated to aid in monitoring treatment, definition and diagnosis of OA as well as new targets for therapeutic drug intervention. These targets were identified by bioinformatic computer analysis and data mining of expressed sequence tags (ESTs) derived from sequencing cDNAs from normal and OA diseased tissues. The data mining effort used sequence comparison techniques (based on BLAST comparison of individual ESTs) to evaluate which ESTs were preferentially observed in the target libraries versus control and/or normal libraries. The selected set of sequences represent a group of genes encoding multiple molecular targets for OA diagnostics. This same set of sequences also represents targets for new OA therapeutic drugs.

Exemplary Uses of the Invention

Useful for generating diagnostic reagents

Nucleic acid probes, proteins, and polypeptides made using these nucleic acids or derived from the sequence information of these nucleic acids are useful for generating diagnostic reagents:

a) The sequences represent markers that correlate with OA disease. Where ESTs are identified as coding for previously identified genes, the full length sequence can be cloned (and appropriate purification tags added if desired), expressed in a bacterial, yeast, insect, mammalian or other cell, and the recombinant protein can be used to raise antibodies which will have utility in OA diagnosis and may aid in analyzing DMOAD efficacy.

b) Sequences representing known genes that are now shown to correlate with OA disease allow commercially produced antibodies to these genes, raised for unrelated purposes, to be evaluated as markers of osteoarthritis disease and their response to drug treatment studied.

c) The full length sequences of the ESTs identified can also be directly injected into animals to elicit an antibody response.

d) Antibodies generated by any of the methods described above can be used to generate ELISAs (Enzyme linked immunosorbent assays) for the detection of OA serum or synovial fluid markers (or other appropriate body tissues and fluids e.g. saliva) and their response to drug treatment. The same antibodies are also useful for detecting OA and DMOAD markers using Western blotting techniques and other antibody-based diagnostics.

e) As described in a) proteins can be generated to the genes represented by the identified ESTs. This process will also generate positive controls to test the sensitivity and efficacy of the ELISAs described in d).

f) Where full length sequences are known or obtained, biochemical assays for the protein product can be designed to generate an assay for OA.

g) The sequences specify oligonucleotides which can be used in polymerase chain reaction (PCR) detection procedures to determine the level of gene expression which infers the cellular state or gene response.

h) PCR primer and probe sets homologous to the identified ESTs can be designed for use in quantitative PCR technology such as the TaqMan technology used in automated fluorescent assays using a variety of large instruments, e.g., LightCycler ABI7700 (Perkin-Elmer), therefore genes identified here as regulated as a result of OA can be quantified in human patients.

i) The sequences can be used to clone their orthologous genes (if present) in species other than Homo sapiens. This allows their expression to be studied in animal models of OA such as the anterior cruciate ligament dog model, the rabbit partial meniscectomy model and the guinea pig spontaneous model.

j) The DNA clones of these sequences can be spotted onto an array or microarray by themselves or as a set or in combination with other genes to determine the level of gene expression which can be used to infer a cellular state or cell response.

k) The sequences can be placed on a computer-readable medium, and can be used to identify another nucleic acid by comparing nucleotide sequence information using computerized means.

Useful as targets for small molecule drug development

The present invention also provides methods and assays to discover compounds and compositions related to OA.

a) The sequences represent genes which can be cloned, over-expressed in a bacterial, yeast, insect, mammalian or other cell, and the active protein used in high throughput screening for novel inhibitors. For example, a biologically-active compound or composition may be identified by examining the interaction between a protein, polypeptide, or fragment of the invention and a compound or composition, and comparing this to a similar interaction of the protein, polypeptide, or fragment of the invention with a control.

b) Compounds and compositions which are detectable through screens using sequences that represent genes are a further embodiment of this invention.

c) The sequences can be used to clone their promoter regions, which in turn can be linked to a reporter gene, such as luciferase, and the resultant recombinant reporter constructs used to screen compounds which alter gene expression.

d) The sequences can be used to identify transcription factors which modulate their expression. These transcription factors can be cloned, over-expressed in a bacterial, yeast, insect, mammalian or other cell, and the active transcription factor used in high throughput screening for small molecule inhibitors of gene expression.

e) The sequences represent cellular markers correlated to a cellular state which individually or in combination can be measured in response to a compound for those compounds which either suppress or activate the genes and thus alter the state of the cell in a desired manner.

Useful for the direct generation of therapeutics

a) The sequences represent genes which can be cloned, over-expressed in a bacterial, yeast, insect, mammalian or other cell, and the encoded protein used as a protein therapeutic.

b) The sequences represent genes which can be directly injected to elicit antibodies useful as therapeutics.

c) The sequences represent genes which can be cloned, expressed in a bacterial, yeast, insect, mammalian or other cell, and the protein used to generate antibodies useful as therapeutics.

d) The sequences can be introduced into an animal to create transgenic animals.

e) The sequences can be used to generate antisense DNA molecules useful to suppress gene expression and provide a therapeutic benefit.

f) The sequences can be used to generate antisense oligonucleotides useful to suppress gene expression and provide a therapeutic benefit.

g) The sequences can be used to generate sense DNA or sense oligonucleotides which will act by co-suppression to provide a therapeutic benefit.

h) The sequences or genes represented by the sequences are useful as gene therapy for activating or suppressing themselves, other genes or entire pathways of genes.

The sequences will facilitate cloning the complete gene

The present invention will facilitate cloning the complete gene, including the entire coding region, the promoter which controls gene transcription, the untranslated region which may control RNA stability and translation as well as for identifying and cloning the genomic clone containing exon and intron information.

a) The sequences specify oligonucleotide templates that can be used to amplify the full length gene using PCR.

b) The DNA clones can be labeled in a manner that they can be used to hybridize to a corresponding full length gene in order to detect and clone the full length gene.

c) The sequences have utility in other procedures, not limited to the two previous examples, to clone the full length gene.

### DETAILED DESCRIPTION OF EXEMPLARY EMBODIMENTS

A. General Concepts and Definitions

General reference texts that provide descriptions of known techniques are discussed herein. These include Current Protocols in Molecular Biology (Ausubel, et al., eds., John Wiley & Sons, N.Y. (1989), and supplements through September 1998), Molecular Cloning, A Laboratory Manual (Sambrook et al., 2nd Ed., Cold Spring Harbor Press, Cold Spring Harbor, N.Y. (1989)), Cells, a Laboratory Manual (Spector et al, eds. Cold Spring Harbor, N.Y. (1998)), and Current Protocols in Immunology (Coligan, ed., John Wiley and Sons, Toronto (1994)), each of which are specifically incorporated by reference in their entirety.

These detailed descriptions are presented for illustrative purposes only and are not intended as a restriction on the scope of the invention. Rather, they are merely some of the embodiments that one skilled in the art would understand from the entire contents of this disclosure. All parts are by weight and temperatures are in Degrees centigrade unless otherwise indicated.

Abbreviations and Definitions

The following is a list of abbreviations and the corresponding meanings as used interchangeably herein:

IMDM=Iscove's modified Dulbecco's media

mg=milligram

ml=milliliter

mL=milliliter

μg=microgram

μl=microliter

ODNs=oligonucleotides

PCR=polymerase chain reaction

RP-HPLC=reverse phase high performance liquid chromatography

ug=microgram

ul=microliter

The following is a list definitions of various terms used herein:

The term “altered” means that expression differs from the expression response of cells or tissues not exhibiting the phenotype.

The term “amino acid(s)” means any of the naturally-occuring alpha-, beta- and gamma-amino carboxylic acids, including their D and L optical isomers and racemic mixtures thereof, and derivatives thereof.

The term “biologically active” means activity with respect to either a structural or a catalytic attribute, which includes the capacity of a nucleic acid to hybridize to another nucleic acid molecule, or the ability of a protein to be bound by an antibody (or to compete with another molecule for such binding), among others. Catalytic attributes involve the capacity of the agent to mediate a chemical reaction or response.

The term “chromosome walking” means a process of extending a genetic map by successive hybridization steps.

The term “cluster” means that BLAST scores from pairwise sequence comparisons of the member clones are similar enough to be considered identical with experimental error.

The term “complement” means that one nucleic acid exhibits complete complementarity with another nucleic acid.

The term “complementarity” means that two molecules can hybridize to one another with sufficient stability to permit them to remain annealed to one another under conventional high stingency conditions.

The term “complete complementarity” means that every nucleotide of one molecule is complementary to a nucleotide of another molecule.

The term “degenerate” means that two nucleic acid molecules encode for the same amino acid sequences but comprise different nucleotide sequences.

The term “exogenous genetic material” means any genetic material, whether naturally occurring or otherwise, from any source that is capable of being inserted into any organism.

The term “expansion” means the differentiation and proliferation of cells.

The term “expressed sequence tags (ESTs)” means randomly sequenced members of a cDNA or complementary DNA library.

The term “expression response” means the mutation affecting the level or pattern of the expression encoded in part or whole by one or more nucleic acid molecules.

The term “fragment” means a nucleic acid (or amino acid) molecule whose sequence is shorter than the target or identified nucleic acid (or amino acid) molecule and having the identical, the substantial complement, or the substantial homologue of at least 10 contiguous nucleotides (or 7 contiguous amino acids) of the target or identified nucleic acid (or amino acid) molecule.

The term “fusion molecule” means a protein-encoding molecule or fragment that upon expression, produces a fusion protein.

The term “fusion protein” means a protein or fragment thereof that comprises one or more additional peptide regions not derived from that protein.

The term “hybridization probe” means any nucleic acid capable of being labeled and forming a double-stranded structure with another nucleic acid over a region large enough for the double stranded structure to be detected.

The term “isolated” means an agent is separated from another specific component with which it occurred. For example, the isolate material may be purified to essential homogeneity, as determined by PAGE or column chromatography, such as HPLC. An isolated nucleic acid can comprise at least about 50, 80, or 90% (on a molar basis) of all macromolecular species present. Some of these methods described later lead to degrees of purification appropriate to identify single bands in electrophoresis gels. However, this degree of purification is not required.

The term “marker nucleic acid” means a nucleic acid molecule that is utilized to determine an attribute or feature (e.g., presence or absence, location, correlation, etc.) of a molecule, cell, or tissue.

The term “mimetic” refers to a compound having similar functional and/or structural properties to another known compound or a particular fragment of that known compound.

The term “minimum complementarity” means that two molecules can hybridize to one another with sufficient stability to permit them to remain annealed to one another under at least conventional low stringency conditions.

The term “PCR probe” means a nucleic acid capable of initiating a polymerase activity while in a double-stranded structure with another nucleic acid. For example, Krzesicki, et al., Am. J. Respir. Cell Mol. Biol. 16:693-701 (1997), incorporated by reference in its entirety, discusses the preparation of PCR probes for use in identifying nucleic acids of osteoarthrits tissue. Other methods for determining the structure of PCR probes and PCR techniques have been described.

The term “phenotype” means any of one or more characteristics of an organism, tissue, or cell.

The term “primer” means a single-stranded oligonucleotide which acts as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template.

The term “probe” means an agent that is utilized to determine an attribute or feature (e.g. presence or absence, location, correlation, etc.) of a molecule, cell, tissue, or organism.

The term “product score” refers to a formula which indicates the strength of a BLAST match using the fraction of overlap of two sequences and the percent identity. The formula is as follows:

$$Product Score = \frac{BLAST Score \times Percent Identity}{5 \times minimum \{ length ( Seq1 ) , length ( Seq2 ) \}}$$

The term “promoter region” means a region of a nucleic acid that is capable, when located in cis to a nucleic acid sequence that encodes for a protein or peptide, of functioning in a way that directs expression of one or more mRNA molecules.

The term “protein fragment” means a peptide or polypeptide molecule whose amino acid sequence comprises a subset of the amino acid sequence of that protein.

The term “protein molecule/peptide molecule” means any molecule that comprises five or more amino acids.

The term “recombinant” means any agent (e.g., DNA, peptide, etc.), that is, or results from, however indirectly, human manipulation of a nucleic acid molecule. The recombination may occur inside a cell or in a tube.

The term “selectable or screenable marker genes” means genes who's expression can be detected by a probe as a means of identifying or selecting for transformed cells.

The term “singleton” means a single clone.

The term “specifically bind” means that the binding of an antibody or peptide is not competitively inhibited by the presence of non-related molecules.

The term “specifically hybridizing” means that two nucleic acid molecules are capable of forming an anti-parallel, double-stranded nucleic acid structure.

The term “substantial complement” means that a nucleic acid sequence shares at least 80% sequence identity with the complement.

The term “substantial fragment” means a fragment which comprises at least 100 nucleotides.

The term “substantial homologue” means that a nucleic acid molecule shares at least 80% sequence identity with another.

The term “substantial identity” means that 70% to about 99% of a region or fragment in a molecule is identical to a region of a different molecule. When the individual units (e.g., nucleotides or amino acids) of the two molecules are schematically positioned to exhibit the highest number of units in the same position over a specific region, a percentage identity of the units identical over the total number of units in the region is determined. Numerous algorithmic and computerized means for determining a percentage identity are known in the art. These means may allow for gaps in the region being considered in order to produce the highest percentage identity.

The term “substantially hybridizing” means that two nucleic acid molecules can form an anti-parallel, double-stranded nucleic acid structure under conditions (e.g. salt and temperature) that permit hybridization of sequences that exhibit 90% sequence identity or greater with each other and exhibit this identity for at least a contiguous 50 nucleotides of the nucleic acid molecules.

The term “substantially purified” means that one or more molecules that are or may be present in a naturally occurring preparation containing the target molecule will have been removed or reduced in concentration.

The term “tissue sample” means any sample that comprises more than one cell.

B. Data Mining and Bioinformatic Computer Analysis

The present invention relates to osteoarthritis tissue-derived nucleic acid sequences SEQ NOs. 1-82. These targets were identified by bioinformatic computer analysis and data mining of expressed sequence tags (ESTs) derived from sequencing cDNAs from normal and OA diseased tissues. The data mining effort used sequence comparison techniques (based on BLAST computer program comparisons of individual ESTs) to evaluate which ESTs were preferentially observed in the target libraries versus control and/or normal libraries. The selected set of sequences represent a group of genes encoding multiple molecular targets for OA diagnostics. This same set of sequences also represents targets for new OA therapeutic drugs.

The present invention also relates to sequences derived from SEQ NOs. 1-82. A variety of computerized means for identifying sequences derived from the SEQ NOs. 1-82 exists. These include the five implementations of BLAST, three designed for nucleotide sequences queries (BLASTN, BLASTX, and TBLASTX) and two designed for protein sequence queries (BLASTP and TBLASTN), as well as FASTA and others (Coulson, Trends in Biotechnology 12:76-80 (1994); Birren et al., Genome Analysis 1:543-559 (1997)).

The BLASTXis one such program that allows the comparison of nucleic acid sequences in the present invention to protein databases to search for the presence of protein homologs. The strength of a BLAST identity match is indicated by the “product score”. This score is a normalized value between 0 and 100, with 100 indicating 100% identity over the entire length of the shorter of the two sequences, and 0 representing no shared identity between the sequences.

Other programs which use either individual sequences or make models from related sequences to further identify sequences derived from SEQ NO 1-SEQ NO 82 exist. Model building and searching programs includes HMer (Eddy), MEME (Bailey and Elkan, Ismb 3: 21-29 (1995)) and PSI-BLAST (Altschul et al., Nucleic Acids Res 25: 3389-3402 (1997)). Another set of programs which use predicted, related, or known protein structures to further identify sequences derived from SEQ NO 1-SEQ NO 82 exists. Structure-based searching programs includes ORF and PROSITE (Bairoch, A. Nucleic Acid Res. 20(Suppl.):2013-18 (1992)). Other programs which use individual sequences or related groups of sequences rely on pattern discovery to further identify sequences derived from SEQ NO:1-82 exist. Pattern recognition programs include Teiresias (Rigoutsos, I. and A. Floratos, Bioinformatics 14:55-67 (1998)), Genotator (Harris, N. Genome Res. 7:754-762 (1997)).

For identification of protein structure, SPASM, RIGOR, and many other programs exist (Kleywegt, G. J. Mol. Biol. 285:1887-97 (1999)). Guermeur et al. Bioinformatics 15:413-21 (1999)). These programs can search any appropriate database, such as but not limited to GenBank, dbEST, EMBL, SwissProt, PIR, and GENES, or protein databases such Protein DataBank (PDB), SCOP, CATH, the Protein Information Resource (PIR) International Protein Sequence Database, the RESAID Database, and FSSP. Computerized means for designing modifications in protein structure are also known in the art (Dahiyat and Mayo, Science 278:82-87 (1997)). Additional information about protein characteristics also can be predicted, such as detecting signal peptides which are secreted (SignalP program) (Nielsen et al., Protein Engineering 12:3-9 (1999)).

The following protein or polypeptide embodiments of the invention can be identified through assays known in the art, including high throughput screening assays. The proteins or polypeptides possess a detectable activity in a functional assay and can be identified by that functional assay. For example, a kinase activity assay is discussed in U.S. Pat. No. 5,759,787, and the references therein.

C. Agents of the Invention

(a) Nucleic Acids

Agents of the present invention include nucleic acids and, more specifically, osteoarthrits tissue-derived nucleic acids. A subset of the nucleic acid molecules of the invention includes nucleic acids that are associated with a gene or fragment thereof. Another subset of the nucleic acids of the invention includes those that encode proteins, polypeptides, or fragments of proteins or polypeptides. In one embodiment, the invention includes nucleic acid sequences having SEQ NOs. 1-82. In one embodiment, the invention includes the sequences identified in Table 1. In another embodiment, the nucleic acids of the invention are derived from one or more EST sequences identified in Table 2.

Fragment nucleic acids may encompass significant portion(s) of, or indeed most of, these nucleic acids. For example, a fragment nucleic acid can encompass an OA marker gene homolog or fragment thereof. Alternatively, the fragments may comprise smaller oligonucleotides (for example, having from about 7 to about 250 nucleotides, or from about 15 to about 30 nucleotide).

Nucleic acids or fragments thereof of the invention are capable of specifically hybridizing to other nucleic acids under certain circumstances. In one embodiment, a nucleic acid of the present invention will specifically hybridize to one or more of the nucleic acids set forth in SEQ NO: 1 through SEQ NO: 82, or complements thereof, under moderately stringent conditions, for example at about 2.0×SSC and about 65° C.

Conventional stringency conditions are described by Sambrook, et al., Molecular Cloning, A Laboratory Manual, 2nd Ed., Cold Spring Harbor Press, Cold Spring Harbor, N.Y. (1989), and by Haymes, et al. Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Ausubel, et al., Current Protocols in Molecular Biology, John Wiley & Sons, N.Y. (1989) (see especially sections 6.3.1-6.3.6). [This reference and the supplements through December 1999 can be relied to make or use any embodiment of the invention.] For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Temperature and salt conditions may be varied independently.

In a particularly preferred embodiment, a nucleic acid of the invention will include those nucleic acids that specifically hybridize to one or more of the nucleic acids set forth in SEQ NO: 1 through SEQ NO: 82, or complements thereof, under high stringency conditions. In one aspect of the present invention, the nucleic acid molecules of the present invention comprise one or more of the nucleic acid sequences set forth in SEQ NO: 1 through to SEQ NO: 82, or complements thereof.

In another aspect of the invention, one or more of the nucleic acid molecules of the present invention share between 80% and 100% sequence identity, or alternatively from between 90% to 100% sequence identity, with one or more of the nucleic acid sequences set forth in SEQ NO: 1 through to SEQ NO: 82 or complements thereof.

(i) Nucleic Acids Comprising Genes or Fragments Thereof

This invention also provides genes corresponding to the cDNA sequences disclosed herein, also called OA Marker nucleic acids. The corresponding genes can be isolated in accordance with known methods using the sequence information disclosed herein. The methods include the preparation of probes or primers from the disclosed sequence information for identification and/or amplification of genes in appropriate genomic libraries or other sources of genomic materials.

The invention provides naturally existing gene homologues or fragments thereof. Genomic sequences can be screened for the presence of protein homologues utilizing one or a number of different search algorithms that have been developed, such as the suite of BLAST programs.

In one embodiment of the present invention, the homologue protein or fragment thereof exhibits a BLASTX probability score of less than 1E-30, or alternatively a BLASTX probability score of between about 1E-30 and about 1E-12, or alternatively a BLASTX probability score of greater than 1E-12 with a nucleic acid or gene of this invention. In another embodiment of the present invention, the nucleic acid molecule encoding the gene homologue or fragment thereof exhibits a % identity with its homologue of between about 25% and about 99%. In a further embodiment, the gene homologue or fragment has a single nucleotide difference from its homologue.

In another preferred embodiment, nucleic acid molecules having SEQ NO: 1 through SEQ NO: 82, or complements and fragments of either, can be utilized to obtain homologues equivalent to the naturally existing homologues. The degeneracy of the genetic code, which allows different nucleic acid sequences to code for the same protein or peptide, is known in the literature (see U.S. Pat. No. 4,757,006). As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate of those set forth in SEQ NO: 1 through to SEQ NO: 82 or complements thereof.

In a further aspect of the present invention, one or more of the nucleic acid molecules of the present invention differ in nucleic acid sequence from those encoding a homologue or fragment thereof in SEQ NO: 1 through SEQ NO: 82 or complements thereof, due to the degeneracy in the genetic code in that they encode the same protein but differ in nucleic acid sequence. In another further aspect of the present invention, one or more of the nucleic acid molecules of the present invention differ in nucleic acid sequence from those encoding an homologue of fragment thereof in SEQ NO: 1 through SEQ NO: 82, or complements thereof, due to fact that the different nucleic acid sequence encodes a protein having one or more conservative amino acid residue. Examples of conservative substitutions are set forth below. Codons capable of coding for such conservative

| Original Residue | Conservative Substitutions |
|---|---|
| Ala | ser |
| Arg | lys |
| Asn | gln; his |
| Asp | glu |
| Cys | ser; ala |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

(ii) Nucleic Acids Comprising Regulatory Elements

One class of agents of the invention includes nucleic acids having promoter regions or partial promoter regions or regulatory elements. Promoter regions are typically found upstream of the trinucleotide ATG sequence at the start site of a protein coding region. The nucleic acids of the invention may be used to isolate promoters of cell-enhanced, cell-specific, tissue-enhanced, tissue-specific, developmentally- or physiologically-regulated expression profiles. Isolation and functional analysis of the 5′ flanking promoter sequences from genomic libraries, for example, using genomic screening methods and PCR techniques, results in the isolation of useful promoters and transcriptional regulatory elements. These methods are known to those of skill in the art and have been described (see, for example, Birren et al., Genome Analysis: Analyzing DNA, 1, (1997), Cold Spring Harbor Laboratory Press, Cold Spring Harbor, N.Y.).

For example, in one embodiment, a regulatory element is detected by incubating nucleic acid(s), or preferably fragments such as ESTs, with members of genomic libraries (e.g., synovial tissue libraries) and recovering clones that hybridize to the nucleic acid(s). Sequencing techniques can then identify regulatory elements from known sequence motifs or known assays for detecting regulatory sequences within a certain proximity to transcription and translation start and stop sites can be used. In a second embodiment, methods of “chromosome walking,” or inverse PCR may be used to obtain regulatory elements (Frohman et al., Proc. Natl. Acad. Sci. (U.S.A.) 85:8998-9002 (1988); Ohara et al., Proc. Natl. Acad. Sci. (U.S.A.) 86: 5673-5677 (1989); Pang et al., Biotechniques 22(6): 1046-1048 (1977); Huang et al., Methods Mol. Biol. 69: 89-96 (1997); Huang, et al., Method Mol. Biol. 67:287-294 (1997); Benkel et al., Genet. Anal. 13: 123-127 (1996); Hartl et al., Methods Mol. Biol. 58: 293-301 (1996)).

Promoters and regulatory elements obtained utilizing the nucleic acids of the invention can also be modified to affect their control characteristics. Examples of these modifications include, but are not limited to, enhancer sequences as reported by Kay et al., Science 236:1299 (1987). Genetic elements such as these can be used to enhance gene expression of new and existing proteins or polypeptides.

(b) Proteins and Polypeptides

This invention also provides a compound or composition comprising one or more polypeptides, which comprise: 1) at least one fragment, segment, or domain of at least 15-1,000 contiguous amino acids, with at least one portion encoded by one or more of SEQ NOS: 1-82; 2) at least one amino acid sequence selected from those encoded by at least one of SEQ NOS: 1-82; or 3) at least one modification corresponding to fragments, segments, or domains within one of SEQ NOS :1-82.

Protein and peptide molecules can be identified using known protein or peptide molecules as a target sequence or target motif in the BLAST programs of the present invention. Proteins or peptides may undergo a variety of modifications, including post-translational modifications, such as disulfide bond formation, glycosylation, phosphorylation, or oligomerization. The term “protein” or polypeptide” includes any protein molecule that is modified by any biological or non-biological process. Proteins encoded by sequences of the present invention can also be fusion proteins. A fusion protein or peptide molecule of the present invention is preferably produced via recombinant means.

Another class of agents comprises protein or peptide molecules encoded by SEQ NO: 1 through SEQ NO: 82 or complements thereof or, fragments or fusions thereof in which conservative, non-essential, or not relevant, amino acid residues have been added, replaced, or deleted. An example is the homologue protein of an OA marker protein. Such a homologue can be obtained by any of a variety of methods. For example, as indicated above, one or more of the disclosed sequences (SEQ NO: 1 through SEQ NO: 82, or complements thereof) will be used to define a pair of primers that may be used to isolate the homologue-encoding nucleic acid molecules from any desired species. Such molecules can be expressed to yield homologs by recombinant means.

Proteins or polypeptides of the invention can be expressed as variants that facilitate purification. For example, a fusion protein to such proteins as maltose binding protein (MBP), glutathione-S-transferase (GST) or thioredoxin (TRX) are known in the art [New England BioLab, Beverly, Mass., Pharmacia, Piscataway, N.J., and InVitrogen, San Diego, Calif.]. The polypeptide or protein can also be a tagged variant to facilitate purification, such as with histidine or methionine rich regions [His-Tag; available from LifeTechnologies Inc, Gaithersburg, Md.] that bind to metal ion affinity chromatography columns, or with an epitope that binds to a specific antibody [Flag, available from Kodak, New Haven, Conn.]. An exemplary, non-limiting list of commercially available vectors suitable for fusion protein expression includes: pBR322 (Promega); pGEX (Amersham); pT7 (USB); pET (Novagen); pIBI (IBI); pProEX-1 (Gibco/BRL); pBluescript II (Stratagene); pTZ18R and pTZ19R (USB); pSE420 (Invitrogen); pAc360 (Invitrogen); pBlueBac (Invitrogen); pBAcPAK (Clontech); pHIL (Invitrogen); pYES2 (Invitrogen); pCDNA (Invitrogen); and pREP (Invitrogen).

A number of other purification methods or means are also known and can be used. These include, but are not limited to, anion or cation exchange chromatography, ethanol precipitation, affinity chromatography, high performance liquid chromatography (HPLC), and reverse-phase high performance liquid chromatography (RP-HPLC), optionally employing hydrophobic RP-HPLC media, e.g., silica gel, to further purify the protein. Combinations of methods and means can also be employed to provide a substantially purified recombinant polypeptide or protein. The particular method used will depend upon the properties of the polypeptide, and the selection of the host cell; appropriate methods will be readily apparent to those skilled in the art.

The polypeptide or protein of the invention may also be expressed via transgenic animals. Methods and means employing the milk of transgenic domestic animals are known in the art.

One or more of the proteins, polypeptides, or fragments may be produced via chemical synthesis. Methods for synthetic construction are known to those skilled in the art. The synthetically-constructed sequences, by virtue of sharing primary, secondary or tertiary structural and/or conformational characteristics with proteins, may possess biological properties in common, including protein activity. Thus, they may be employed as biologically active or immunological substitutes for natural, purified proteins in screening of therapeutic compounds and in immunological processes for the development of antibodies.

(c) Antibodies

One aspect of the present invention concerns antibodies, single-chain antigen binding molecules, or other proteins that specifically bind to one or more of the protein or peptide molecules of the present invention and their homologues, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules.

Nucleic acid molecules that encode all or part of the protein of the present invention can be expressed, by recombinant means, to yield protein or peptides that can in turn be used to elicit antibodies that are capable of binding the expressed protein or peptide. Such antibodies may be used in immunoassays for that protein. Such protein-encoding molecules or their fragments may be a “fusion” molecule (i.e., a part of a larger nucleic acid molecule) such that, upon expression, a fusion protein is produced. It is understood that any of the nucleic acid molecules of the present invention may be expressed, by recombinant means, to yield proteins or peptides encoded by these nucleic acid molecules.

The antibodies that specifically bind proteins and protein fragments of the present invention may be polyclonal or monoclonal, and may comprise intact immunoglobulins, or antigen binding portions of immunoglobulins (such as (F(ab′), F(ab′)₂ fragments), or single-chain immunoglobulins producible, for example, via recombinant means. Conditions and procedures for the construction, manipulation and isolation of antibodies (see, for example, Harlow and Lane, Antibodies: A Laboratory Manual, Cold Spring Harbor Press, Cold Spring Harbor, N.Y. (1988)) are well known in the art.

Additionally, methods of raising antibodies to polypeptides of the present invention are well known in the art (Harlow and Lane, Antibodies: A Laboratory Manual, Cold Spring Harbor Press, Cold Spring Harbor, N.Y. (1988)). Animals are immunized with purified protein (or fragment thereof) in appropriate amounts and using standard schedules, and are bled and the blood screened for the presence of anti-protein or peptide antibodies using, for example, a direct binding Enzyme-Linked Immunoassay (ELISA). In one embodiment, anti-protein or peptide monoclonal antibodies are isolated using a fusion of a protein, protein fragment, or peptide of the present invention, or conjugate of a protein, protein fragment, or peptide of the present invention, as immunogens. It is understood that any of the protein or peptide molecules of the present invention may be used to raise antibodies.

As discussed below, such antibody molecules or their fragments may be used for diagnostic purposes. Where the antibodies are intended for diagnostic purposes, it may be desirable to derivatize them, for example with a ligand group (such as biotin) or a detectable marker group (such as a fluorescent group, a radioisotope or an enzyme).

The ability to produce antibodies that bind the protein or peptide molecules of the present invention permits the identification of mimetic compounds of those molecules. Mimetic compounds can be synthesized chemically. Combinatorial chemistry techniques, for example, can be used to produce libraries of peptides (see WO 9700267), polyketides (see WO 960968), peptide analogues (see WO 9635781, WO 9635122, and WO 9640732), oligonucleotides for use as mimetic compounds derived from this invention. Mimetic compounds and libraries can also be generated through recombinant DNA-derived techniques. For example, phage display libraries (see WO 9709436), DNA shuffling (see U.S. Pat. No. 5,811,238) other directed or random mutagenesis techniques can produce libraries of expressed mimetic compounds. It is understood that any of the agents of the present invention can be substantially purified and/or be biologically active and/or recombinant.

(d) Transformed Cells

A recombinant protein may be produced by opererably linking a regulatory control sequence to a nucleic acid of the present invention and putting it into an expression vector. Regulatory sequences include promoters, enhancers, and other expression control elements which are described in Goeddel (Hene Expression Technology: Methods in Enzymology 185. Academic Press, San Diego, Calif. (1990)). For example, the native regulatory sequences or regulatory sequences native to the transformed host cell can be used. One of skill in the art is familiar with numerous examples of these additional functional sequences, as well as other functional sequences, that may optionally be included in an expression vector. The design of the expression vector may depend on such factors as the choice of the host cell to be transformed, and/or the type of protein desired. Many such vectors are commercially available, including linear or enclosed elements (see for example, Broach, et al., Experimental Manipulation of Gene Expression, ed. M. Inouye, Academic Press, (1983); Sambrook, et al., Molecular Cloning, A Laboratory Manual, 2nd Ed., Cold Spring Harbor Press, Cold Spring Harbor, N.Y. (1989)). Typically, expression constructs will contain one or more selectable markers, including the gene that encodes dihydrofolate reductase and the genes that confer resistance to neomycin, tetracycline, ampicillin, chloramphenicol, kanamycin and streptomycin resistance.

Prokaryotic and eukaryotic host cells transfected by the described vectors are also provided by this invention. For instance, cells which can be transfected with the vectors of the present invention include, but are not limited to, bacterial cells such as E. coli (e.g., E. coli K 12 strains), Streptomyces, Pseudomonas, Serratia marcescens and Salmonella typhimurium, insect cells (baculovirus), including Drosophila, fungal cells, such as yeast cells, plant cells, and ovary cells (CHO), and COS cells.

Thus, the nucleic acid molecules described can be used to produce a recombinant form of the protein via microbial or eukaryotic cellular processes. Ligating the polynucleic acid molecule into a gene construct, such as an expression vector, and transforming or transfecting into hosts, either eukaryotic (yeast, avian, insect, plant, or mammalian) or prokaryotic (bacterial cells), are standard procedures used in producing other well known proteins. Similar procedures, or modifications thereof, can be employed to prepare recombinant proteins according to the present invention by microbial means or tissue-culture technology. Accordingly, the invention pertains to the production of encoded proteins or polypeptides by recombinant technologies.

D. Uses of the Agents of the Invention

1. Methods for Identifying Bioactive Proteins, Polypeptides, Fragments, or Variants of the Invention

Once the nucleic acid has been used to produce a protein, polypeptide, or a variant or fragment, any one of a number of assays can be used to identify bioactivity.

In addition, the agents of the invention are especially useful in high throughput screening methods. In general, these methods involve individual sample assay volumes less than about 250 μl, or more preferably less than about 100 μl. With smaller sample volumes, numerous individual assays can be performed simultaneously and via computer-operated instrumentation. The assays comprise the detectable interaction between a protein, polypeptide, fragment, nucleic acid, or antibody of the invention (sometimes referred to as the target) and an assay compound. Thus, the assays comprise two components, a target and an assay compound, where the assay compound may be part of a composition of multiple compounds. It is also possible for the agents of this invention to be used as assay compounds in screening methods where other proteins, polypeptides, nucleic acids, antibodies, or binding partners are the targets.

The assay compound can be selected from a library of small molecules, organic compounds which are either synthetic or natural, or mimetic libraries of randomized oligonucleotide-derived or peptide-derived compounds, for example. The compounds of the libraries may contain random chemical modifications, such as acylation, alkylation, esterification, amidation, or other modifications. Ideally, the largest number of separate structural entities will exist in a library that is tested against the agents of the invention for detectable interaction. A variety of other reagents may be used in the assay, such as buffers, salts, detergents, proteins, protease inhibitors, nuclease inhibitors, antimicrobial agents, or other reagents.

Detecting the interaction between the assay compound and the agent of the invention can be performed via a number of techniques. Fluorescence quenching, specific binding as with avidin-biotin, enzymatic activity, or inhibition of enzymatic activity are examples of the types of techniques used to detect interaction between two molecules. One of skill in the art can devise many specific assays depending on the activity sought. The type of assay used is not crucial to the use of this invention.

The screening methods may optionally employ a solid substrate to which one or more assay components are bound. Also, cell-based assays are often used in high throughput screening methods, so that the cell contains or expresses a component of the assay. Numerous permutations are possible.

Each of the activities listed below may be screened for, alone or in combination, in a method to detect an interaction with agents of the invention.

a. Inflammatory or Anti-Inflammatory Activity

Proteins or polypeptides of the present invention may also exhibit inflammatory or anti-inflammatory activity. These activities may relate to a stimulus to cells involved in the inflammatory response, inhibiting or promoting cell-cell interactions (for example, cell adhesion), inhibiting or promoting chemotaxis of cells involved in the inflammatory process, inhibiting or promoting cell extravasation, or stimulating or suppressing production of other factors, which more directly inhibit or promote an inflammatory response.

Proteins or polypeptides exhibiting anti-inflammatory activity or antibodies to inflammatory proteins or polypeptides can be used to treat atopic disorders and other inflammatory conditions including: chronic or acute inflammatory conditions, inflammation associated with infection, such as septic shock, sepsis or systemic inflammatory response syndrome (SIRS); ischemia-reperfusion injury; endotoxin lethality; arthritis; complement-mediated hyperacute rejection; nephritis; cytokine or chemokine-induced lung injury; inflammatory bowel disease; Crohn's disease; or disorders resulting from over-production of cytokines such as TNF or IL-1. Proteins or polypeptides or antibodies of the invention may also be useful to treat anaphylaxis and hypersensitivity to an antigenic substance or material.

b. Cytokine and Cell Growth or Differentiation Activity

A protein or polypeptide of the invention may exhibit cytokine, cell growth promoting or inhibiting, or cell differentiation promoting or inhibiting activity. Many protein factors secreted by immune cells, including cytokines, have exhibited activity in one or more factor dependent cell-based assays. These assays can be used to identify useful activities. The activity of a protein or polypeptide of the invention may be measured by the following methods or others known in the art.

Assays for T-cell or thymocyte proliferation include those described in Current Protocols in Immunology, Coligan, et al. Eds., Greene Publishing Associates and Wiley-Interscience (1994) (see, especially Chapter 3, In Vitro assays for Mouse Lymphocyte Function 3.1-3.19; Chapter 7, Immunologic studies in Humans); Takai et al., J. Immunol. 137:3494-3500 (1986); Bertagnolli et al., J. Immunol. 145:1706-1712 (1990); Bertagnolli et al., Cellular Immunology 133:327-341 (1991); Bertagnolli, et al., J. Immunol. 149:3778-3783 (1992); Bowman et al., J. Immunol. 152: 1756-1761 (1994)).

Assays for cytokine production and/or proliferation of spleen cells, lymph node cells, or thymocytes include those described in Polyclonal T cell stimulation, Kruisbeek, and Shevach, In Current Protocols in Immunology, Coligan eds. Vol 1 pp. 3.12.1-3.12.14, John Wiley and Sons, Toronto (1994); and Measurement of mouse and human Interferon gamma, Schreiber, In Current Protocols in Immunology, Coligan eds. Vol 1 pp. 6.8.1-6.8.8, John Wiley and Sons, Toronto (1994).

Assays for proliferation and differentiation of hematopoietic and lymphopoietic cells include those described in Measurement of Human and Murine Interleukin 2 and Interleukin 4, Bottomly, Davis, and Lipsky In Current Protocols in Immunology. Coligan eds. Vol 1 pp. 6.3.1-6.3.12, John Wiley and Sons, Toronto (1994); deVries et al., J. Exp. Med. 173:1205-1211 (1991); Moreau et al., Nature 336:690-692 (1988); Greenberger et al., Proc. Natl. Acad. Sci. (U.S.A.) 80:2931-2938 (1983); Measurement of mouse and human interleukin 6, Nordan, R. In Current Protocols in Immunology. Coligan eds. Vol 1 pp. 6.6.1-6.6.5, John Wiley and Sons, Toronto (1994); Smith et al., Proc. Natl. Acad. Sci. (U.S.A.) 83:1857-1861 (1986); Measurement of human Interleukin 11, Bennett, et al., In Current Protocols in Immunology. Coligan eds. Vol 1 pp. 6.15.1 John Wiley and Sons, Toronto (1991); Measurement of mouse and human Interleukin 9, Ciarletta, et al., In Current Protocols in Immunology. Coligan eds. Vol 1 pp. 6.13.1, John Wiley and Sons, Toronto (1991).

Assays for T-cell clone responses to antigens (which will identify, among others, proteins that affect antigen-presenting cell (APC)-T cell interactions as well as direct T-cell effects by measuring proliferation and cytokine production) include, without limitation, those described in: Current Protocols in Immunology, Coligan, et al. eds., Pub. Greene Publishing Associates and Wiley-Interscience (1994)(Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. (U.S.A) 77:6091-6095, 1980; Weinberger et al., Eur. J. Immun. 11:405-411, 1981; Takai et al., J. Immunol. 137:3494-3500, 1986; Takai et al., J. Immunol. 140:508-512 (1988).

c. Immunosuppressive, Immune Stimulating, or Immune Modulating Activity

Suitable assays for thymocyte or splenocyte cytotoxicity include, without limitation, those described in: Current Protocols in Immunology, Ed by Coligan, et al., Pub. Greene Publishing Associates and Wiley-Interscience (1994)(Chapter 3, In Vitro assays for Mouse Lymphocyte Function 3.1-3.19; Chapter 7, Immunologic studies in Humans); Herrmann et al., Proc. Natl. Acad. Sci. (U.S.A.) 78:2488-2492 (1981); Herrmnann et al., J. Immunol. 128:1968-1974 (1982); Handa et al., J. Immunol. 135:1564-1572 (1985); Takai et al., J. Immunol. 137:3494-3500 (1986); Takai et al., J. Immunol. 140:508-512 (1988); Bowman et al., J. Virology 61:1992-1998; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028:3033 (1990); and Assays for B cell function: In vitro antibody production, Mond, and Brunswick, In Current Protocols in Immunology. Coligan eds. Vol 1 pp. 3.8.1-3.8.16, John Wiley and Sons, Toronto (1994).

Mixed lymphocyte reaction (MLR) assays (which will identify, among others, proteins that generate predominantly Th1 and CTL responses) include, without limitation, those described in: Current Protocols in Immunology, Ed by Coligan, et al., Strober, Pub. Greene Publishing Associates and Wiley-Interscience (1994) (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783 (1992).

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., J Immunol 154:5071-5079 (1995); Porgador et al., Journal of Experimental Medicine 182:255-260 (1995); Nair et al., J. Virology 67:4062-4069 (1993); Huang et al., Science 264:961-965 (1994); Macatonia et al., Journal of Experimental Medicine 169:1255-1264 (1989); Bhardwaj et al., Journal of Clinical Investigation 94:797-807 (1994); and Inaba et al., Journal of Experimental Medicine 172:631-640 (1990).

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808 (1992); Gorczyca et al., Leukemia 7:659-670 (1993); Gorczyca et al., Cancer Research 53:1945-1951 (1993); Itoh et al., Cell 66:233-243 (1991); Zacharchuk, J. Immunol. 145:4037-4045 (1990); Zamai et al., Cytometry 14:891-897 (1993); Gorczyca et al., International Journal of Oncology 1:639-648 (1992).

Assays for proteins that influence early steps of T-cell commitment and development include, without limitation, those described in: Antica et al., Blood 84:111-117 (1994); Fine et al., Cellular Immunology 155:111-122 (1994); Galy et al., Blood 85:2770-2778 (1995); Toki et al., Proc. Nat. Acad Sci. (U.S.A.) 88:7548-7551 (1991).

d. Cell Differentiation Activity

Assays for embryonic stem cell differentiation (which will identify, among others, proteins that influence embryonic differentiation hematopoiesis) include, without limitation, those described in: Johansson et al., Cellular Biology 15:141-151 (1995); Keller et al., Molecular and Cellular Biology 13:473-486 (1993); McClanahan et al., 81:2903-2915 (1993).

Assays for stem cell survival and differentiation (which will identify, among others, proteins that regulate lympho-hematopoiesis) include, without limitation, those described in: Methylcellulose colony forming assays, Freshney, In Culture of Hematopoietic Cells. Freshney, et al., eds. Vol pp. 265-268, Wiley-Liss, Inc., New York, N.Y. (1994); Hirayama et al., Proc. Natl. Acad. Sci. (U.S.A.) 89:5907-5911 (1992); Primitive hematopoietic colony forming cells with high proliferate potential, McNiece, and Briddell, In Culture of Hematopoietic Cells, Freshney, et al. eds. Vol pp. 23-39, Wiley-Liss, Inc., New York, N.Y. (1994); Neben et al., Experimental Hematology 22:353-359 (1994); Cobblestone area forming cell assay, Ploemacher, In Culture of Hematopoietic Cells, Freshney, et al., eds. Vol pp. 1-21, Wiley-Liss, Inc., New York, N.Y. (1994); and Long term bone marrow cultures in the presence of stromal cells, Spooncer, et al., In Culture of Hematopoietic Cells, Freshney, et al., eds. Vol pp. 163-179, Wiley-Liss, Inc., New York, N.Y. (1994); Long term culture initiating cell assay, Sutherland, In Culture of Hematopoietic Cells, R. I. Freshney, et al., eds. Vol pp. 139-162, Wiley-Liss, Inc., New York, N.Y. (1994).

e. Wound Healing Activity

Assays for tissue generation activity include, without limitation, those described in: International Patent Publication No. WO95/16035 (bone, cartilage, tendon); International Patent Publication No. WO95/05846 (nerve, neuronal); and International Patent Publication No. WO91/07491 (skin, endothelium). Assays for wound healing activity include, without limitation, those described in: Winter, Epidermal Wound Healing, pps. 71-112 (Maibach, and Rovee, eds.), Year Book Medical Publishers, Inc., Chicago, as modified by Eaglstein and Mertz, J. Invest. Dermatol 71:382-84 (1978).

f. Chemotactic Activity

A protein or peptide has chemotactic activity for a particular cell population if it can stimulate, directly or indirectly, the controlled orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by Coligan et al., Pub. Greene Publishing Associates and Wiley-Interscience (Chapter 6.12, Measurement of alpha and beta Chemokines 6.12.1-6.12.28; Taub et al., J. Clin. Invest. 95:1370-1376 (1995); Lind et al., APMIS 103:140-146 (1995); Muller et al., Eur. J. Immunol. 25: 1744-1748; Gruber et al., J. of Immunol. 152:5860-5867 (1994); and Johnston et al. J. of Immunol. 153: 1762-1768 (1994).

g. Receptor/Ligand Interaction Activity

Proteins of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). These proteins or fragments of the invention, or cells containing them, can be incorporated into an assay to screen for binding to extracellular matrix proteins, their analogs, or for receptor/ligand interaction to compounds implicated in binding to extracellular matrix proteins. In a particularly preferred embodiment, molecules containing the peptide motif RGD can be used to screen for interaction with the proteins, fragments, or cells of the invention. Various specific assays can be used a basis for designing the reagents for screening, such as phage attachment assays, panning assays, cell attachment assays, and inhibition of cell attachment/adhesion assays (Pasquelina et al., J. Cell Biol. 130:1189-1196 (1995), Koivunen et al., BioTechnology 13: 265-270 (1995), Koivunen et al., Methods Enzym. 245: 346-369 (1994), and U.S. Pat. No. 5,817,750, all incorporated herein in their entirety). These receptor/ligand interaction assays can also be designed for use with libraries of compounds, such as phage display libraries.

Receptors and ligands are also useful for screening of potential peptide or small molecule inhibitors of the relevant receptor/ligand interaction. Proteins of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by Coligan, et al., Pub. Greene Publishing Associates and Wiley-Interscience (Chapter 7.28, Measurement of Cellular Adhesion under Static Conditions 7.28.1-7.28.22); Takai et al., Proc. Natl. Acad. Sci. (U.S.A.) 84:6864-6868 (1987); Bierer et al., J. Exp. Med. 168:1145-1156 (1988); Rosenstein et al., J. Exp. Med. 169:149-160 (1989); Stoltenborg et al., J. Immunol. Methods 175:59-68, 1994; Stitt et al., Cell 80:661-670 (1995).

2. Methods for Detecting and Manipulating Nucleic Acids

The nucleic acids of the invention can be used directly in numerous methods to identify or detect the presence of specific nucleic acid sequences. As noted above, the nucleic acids of the invention can be used as hybridization probes or PCR probes, or to derive specific hybridization or PCR probes. Agents of the present invention may be labeled with reagents that facilitate detection (e.g., fluorescent labels, Prober et al., Science 238: 336-340 (1987), Albarella et al., EP 144914;, chemical labels, Sheldon et al., U.S. Pat. No. 4,582,789, Albarella et al., U.S. Pat. No. 4,563,417; and modified bases, Miyoshi et al., EP 119448)).

Furthermore, the nucleic acids of the invention of variants or fragments thereof can be linked to solid supports. In this way, various microarrays, beads, glass or nylon slides, membranes or other repeatable assay apparati can be constructed. A non-limiting description of selected methods follows.

a. Microarrays

In one embodiment, the nucleic acids of the invention can be used to monitor expression. A microarray-based method for high-throughput monitoring of gene expression may be utilized to measure activated OA Marker hybridization targets. This ‘chip’-based approach involves using microarrays of nucleic acids as specific hybridization targets to quantitatively measure expression of the corresponding genes (Schena et al., Science 270:467-470 (1995); Shalon, Ph.D. Thesis, Stanford University (1996). Every nucleotide in a large sequence can be queried at the same time. Hybridization can also be used to efficiently analyze nucleotide sequences.

Several microarray methods have been described. One method compares the sequences to be analyzed by hybridization to a set of oligonucleotides or cDNA molecules representing all possible subsequences (Bains and Smith, J. Theor. Biol. 135:303 (1989)). A second method hybridizes the sample to an array of oligonucleotide or cDNA probes. An array consisting of oligonucleotides or cDNA molecules complementary to subsequences of a target sequence can be used to determine the identity of a target sequence, measure its amount, and detect differences between the target and a reference sequence. Nucleic acid microarrays may also be screened with protein molecules or fragments thereof to determine nucleic acids that specifically bind protein molecules or fragments thereof.

The microarray approach may also be used with polypeptide targets (see, U.S. Pat. Nos. 5,800,992, 5,445,934; 5,143,854, 5,079,600, 4,923,901). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides (Fodor et al., Science 251:767-773 (1991)).

b. Hybridization Assays

Oligonucleotide probes, whose sequences are complementary to that of a portion of the nucleic acids of the invention, such as SEQ NO.: 1-82, can be constructed. These probes are then incubated with cell extracts of a patient under conditions sufficient to permit nucleic acid hybridization. The detection of double-stranded probe-mRNA hybrid molecules is indicative of the presence of OA Markers or sequences derived from OA Markers. Thus, such probes may be used to ascertain the level and extent of OA severity or progression or the production of certain proteins. The nucleic acid hybridization may be conducted under quantitative conditions or as a qualitative assay.

c. PCR Assays

A nucleic acid of the invention, such as one of SEQ NO.: 1-82 or complements thereof, can be analyzed for use as a PCR probe. A search of databases indicates the presence of regions within that nucleic acid that have high and low regions of identity to other sequences in the database. Ideally, a PCR probe will have high identity with only the sequence from which it is derived. In that way, only the desired sequence is amplified. Computer generated searches using programs such as MIT Primer3 (Rozen and Skaletsky (1996, 1997, 1998)) , or GeneUp (Pesole, et al., BioTechniques 25:112-123 (1998)), for example, can be used to identify potential PCR primers.

The PCR probes or primers can be used in methods such as described in Krzesicki, et al., Am. J. Respir. Cell Mol. Biol. 16:693-701 (1997) to identify or detect sequences expressed in osteoarthritis.

d. Ligation and Alternative Amplification Methods and Identification of Polymorphisms

In one sub-aspect of the invention analysis is conducted to determine the presence and/or identity of polymorphism(s) using one or more of the nucleic acid molecules of the present invention and more specifically one or more of the EST nucleic acid molecule or fragment thereof which are associated with a phenotype, or a predisposition to that phenotype.

Any of a variety of molecules can be used to identify such polymorphism(s). In one embodiment, one or more of the EST nucleic acid molecules (or a sub-fragment thereof) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

The genomes of animals and plants naturally undergo spontaneous mutation in the course of their continuing evolution, creating polymorphisms (Gusella, Ann. Rev. Biochem. 55:831-854 (1986)). The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a di-allelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour, et al., FEBS Lett. 307:113-115 (1992); Jones, et al., Eur. J. Haematol. 39:144-147 (1987); Horn, et al., PCT Patent Application WO91/14003; Jeffreys, European Patent Application 370,719; Jeffreys, U.S. Pat. No. 5,175,082; Jeffreys et al., Amer. J. Hum. Genet. 39:11-24 (1986); Jeffreys et al., Nature 316:76-79 (1985); Gray et al., Proc. R. Acad. Soc. Lond. 243:241-253 (1991); Moore et al., Genomics 10:654-660 (1991); Jeffreys et al., Anim. Genet. 18:1-15 (1987); Hillel et al., Anim. Genet. 20:145-155 (1989); Hillel et al., Genet. 124:783-789 (1990)).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”)(See above). In lieu of PCR, alternative amplification methods, such as the “Ligase Chain Reaction” (“LCR”) may be used (Barany, Proc. Natl. Acad. Sci. (U.S.A.) 88:189-193 (1991). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069).

The “Oligonucleotide Ligation Assay” (“OLA”) may alternatively be employed (Landegren, et al., Science 241:1077-1080 (1988)). The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, et al. have described a nucleic acid detection assay that combines attributes of PCR and OLA (Nickerson, et al., Proc. Natl. Acad. Sci. (U.S.A.) 87:8923-8927 (1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide,” thereby amplifying the di-oligonucleotide, are also known (Wu, et al., Genomics 4:560 (1989)), and may be readily adapted to the purposes of the present invention. Other known nucleic acid amplification procedures, such as allele-specific oligomers, branched DNA technology, transcription-based amplification systems, or isothermal amplification methods may also be used to amplify and analyze such polymorphisms (Malek, et al., U.S. Pat. No. 5,130,238; Davey, et al., European Patent Application 329,822; Schuster et al., U.S. Pat. No. 5,169,766; Miller, et al., PCT appln. WO 89/06700; Kwoh, et al., Proc. Natl. Acad. Sci. (U.S.A.) 86:1173 (1989); Gingeras, et al., PCT application WO 88/10315; Walker, et al., Proc. Natl. Acad. Sci. (U.S.A.) 89:392-396 (1992)). Any of the foregoing nucleic acid amplification methods could be used.

The identification of a polymorphism in a gene, fragment or cellular sequence derived from the nucleic acids of the invention can be determined in a variety of ways. By correlating the presence or absence of atopic disease, for example, in an individual with the presence or absence of a polymorphism, it is possible to diagnose the predisposition of a patient to osteoarthritis-related disorders. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and animal genetic analyses (Glassberg, UK Patent Application 2135774; Skolnick, et al., Cytogen. Cell Genet. 2 5 32:58-67 (1982); Botstein, et al., Ann. J. Hum. Genet. 32:314-331 (1980); Fischer, et al. (PCT Application WO90/13668); Uhlen, PCT Application WO90/11369).

Other types of polymorphisms include single nucleotide polymorphisms (SNPs) that are single base changes in genomic DNA sequence. They generally occur at greater frequency than other markers and are spaced with a greater uniformity throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a result of mutations or defective splicing). Analysis (genotyping) of characterized SNPs can require only a plus/minus assay rather than a lengthy measurement, permitting easier automation.

Modifications can be naturally provided or deliberately engineered into the nucleic acids, proteins, and polypeptides of the invention to generate variants. For example, modifications in the peptide or DNA sequences can be made by those skilled in the art using known techniques, such as site-directed mutagenesis. Modifications of interest in the protein sequences may include the alteration, substitution, replacement, insertion or deletion of one or more selected amino acid residues. For example, one or more cysteine residues may be deleted or replaced with another amino acid to alter the conformation of the molecule. Additional cysteine residues can also be added as a substitute at sites to promote disulfide bonding and increase stability. Techniques for identifying the sites for alteration, substitution, replacement, insertion or deletion are well known to those skilled in the art. Techniques for making alterations, substitutions, replacements, insertions or deletions (see, e.g., U.S. Pat. No. 4,518,584) are also well known in the art. Preferably, any modification of a protein, polypeptide, or nucleic acid of the invention will retain at least one of the structural or functional attributes of the molecule.

SNPs can be characterized using any of a variety of methods. Such methods include the direct or indirect sequencing of the site, the use of restriction enzymes (Botstein et al., Am. J. Hum. Genet. 32:314-331 (1980)); Konieczny and Ausubel, Plant J. 4:403-410 (1993), enzymatic and chemical mismatch assays (Myers et al., Nature 313:495-498 (1985), allele-specific PCR (Newton et al., Nucl. Acids Res. 17:2503-2516 (1989)); Wu et al., Proc. Natl. Acad. Sci. USA 86:2757-2760 (1989)), ligase chain reaction (Barany, Proc. Natl. Acad. Sci. USA 88:189-193 (1991)), single-strand conformation polymorphism analysis (Labrune et al., Am. J. Hum. Genet. 48: 1115-1120 (1991)), primer-directed nucleotide incorporation assays (Kuppuswami et al., Proc. Natl. Acad. Sci. USA 88:1143-1147 (1991)), dideoxy fingerprinting (Sarkar et al., Genomics 13:441-443 (1992)), solid-phase ELISA-based oligonucleotide ligation assays (Nikiforov et al., Nucl. Acids Res. 22:4167-4175 (1994)), oligonucleotide fluorescence-quenching assays (Livak et al., PCR Methods Appl. 4:357-362 (1995a)), 5′-nuclease allele-specific hybridization TaqMan assay (Livak et al., Nature Genet. 9:341-342 (1995)), template-directed dye-terminator incorporation (TDI) assay (Chen and Kwok, Nucl. Acids Res. 25:347-353 (1997)), allele-specific molecular beacon assay (Tyagi et al., Nature Biotech. 16: 49-53 (1998)), PinPoint assay (Haff and Smirnov, Genome Res. 7: 378-388 (1997)), and dCAPS analysis (Neff et al., Plant J. 14:387-392 (1998)).

SNPs can be observed by examining sequences of overlapping clones in the BAC library according to the method described by Taillon-Miller et al. Genome Res. 8:748-754 (1998)). SNPs can also be observed by screening the BAC library of the present invention by colony or plaque hybridization with a labeled probe containing SNP markers; isolating positive clones and sequencing the inserts of the positive clones; suitable primers flanking the SNP markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5:874-879 (1989)). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to, Lee et al., Anal. Biochem. 205:289-293 (1992); Suzuki et al., Anal. Biochem. 192:82-84 (1991); Lo et al., Nucleic Acids Research 20: 1005-1009 (1992); and Sarkar et al., Genomics 13:441-443 (1992). It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos et al., Nucleic Acids Res. 23:4407-4414 (1995)). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann et al., Mol. Ecol. 6:989-993 (1997)), Acinetobacter (Janssen et al., Int. J. Syst. Bacteriol. 47:1179-1187 (1997)), Aeromonas popoffi (Huys et al., Int. J. Syst. Bacteriol. 47:1165-1171 (1997)), Phytophthora infestans (Van der Lee et al., Fungal Genet. Biol. 21:278-291 (1997)), Bacillus anthracis (Keim et al., J. Bacteriol. 179:818-824 (1997)), Astragalus cremnophylax (Travis et al., Mol. Ecol. 5:735-745 (1996)), Arabidopsis (Cnops et al., Mol. Gen. Genet. 253:32-41 (1996)), Escherichia coli (Lin et al., Nucleic Acids Res. 24:3649-3650 (1996)), Aeromonas (Huys et al., Int. J. Syst. Bacteriol. 46:572-580 (1996)), nematode (Folkertsma et al., Mol. Plant Microbe Interact. 9:47-54 (1996)), and human (Latorra et al., PCR Methods Appl. 3:351-358 (1994)). AFLP analysis has also been used for fingerprinting mRNA (Money et al., Nucleic Acids Res. 24:2616-2617 (1996), the entirety of which is herein incorporated by reference; Bachem et al., Plant J. 9:745-753 (1996)). It is understood that one or more of the nucleic acids of the present invention, may be utilized as markers or probes to detect polymorphisms by AFLP analysis or for fingerprinting RNA.

The polymorphism obtained by these approaches can then be cloned to identify the mutation at the coding region, which alters the protein's structure or the regulatory region of the gene that affects its expression level. Changes involving promoter interactions with other regulatory proteins can be identified by, for example, gel shift.

In accordance with an embodiment of the invention, a sample DNA is obtained from a patient's cells. In a preferred embodiment, the DNA sample is obtained from the patient's blood. However, any source of DNA may be used. The DNA may be subjected to interrogation to determine the presence or absence of a polymorphism.

3. Screening Assays

The invention provides a method (also referred to herein as a “screening assay”) for identifying modulators, i.e., candidate or test compounds or agents (e.g., antisense, polypeptides, peptidomimetics, small molecules or other drugs) which bind to nucleic acid molecules, polypeptides or proteins described herein or have a stimulatory or inhibitory effect on, for example, expression or activity of the nucleic acid molecules, polypeptides or proteins of the invention.

In one embodiment, the invention provides assays for screening candidate or test compounds which bind to or modulate the activity of protein or polypeptide described herein or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. U.S.A., 90:6909; Erb et al. (1994) Proc. Natl. Acad. Sci. U.S.A., 91:11422; Zuckermann et al. (1994). J.

Med. Chem., 37:2678; Cho et al. (1993) Science, 261:1303; Carell et al. (1994) Angew. Chem. Int. Ed. Engl., 33:2059; Carell et al. (1994) Angew. Chem. Int. Ed. Engl., 33:2061; and in Gallop et al. (1994) J. Med. Chem., 37:1233.

Libraries of compounds may be presented in solution (e.g., Houghten (1992) Biotechniques, 13:412-421), or on beads (Lam(1991) Nature, 354:82-84), chips (Fodor (1993) Nature, 364;555-556), bacteria (Ladner U.S. Pat. No. 5,223,409), spores (Ladner U.S. Pat. No. 409), plasmids (Cull et al.(1992) Proc. Natl. Acad. Sci. U.S.A., 89:1865-1869) or on phage (Scott and Smith (1990) Science, 249:386-390); (Devlin (1990) Science, 249:404-406); (Cwirla et al. (1990) Proc. Natl. Acad. Sci., 97:6378-6382); (Felici (1991) J. Mol. Biol., 222:301-310); (Ladner supra).

In one embodiment, an assay is a cell-based assay in which a cell which expresses an encoded protein which is a cell surface receptor is contacted with a test compound and the ability of the test compound to bind to the receptor is determined. The cell, for example, can be of mammalian origin, such as from cartilage tissue or bone. Determining the ability of the test compound to bind to the receptor can be accomplished, for example, by coupling the test compound with a radioisotope or enzymatic label such that binding of the test compound to the receptor can be determined by detecting the labeled with .sup.125 I, .sup.35 S, sup. 14 C, or .sup.3 H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with the receptor without the labeling of any of the interactants. For example, a microphysiometer can be used to detect the interaction of a test compound with the receptor without the labeling of either the test compound or the receptor. McConnell, H. M. et al. (1992) Science, 257:1906-1912. A “microphysiometer” (e.g., Cytosensor.TM.) is an analytical instrument that measures the rate at which a cell acidifies its environment using a light-addressable potentiometric sensor (LAPS). Changes in this acidification rate can be used as an indicator of the interaction between ligand and receptor.

In one embodiment, the assay comprises contacting a cell which expresses an encoded protein described herein on the cell surface (e.g., a receptor) with a receptor ligand or biologically-active portion thereof, to form an assay mixture, contacting the assay mixture with a test compound, and determining the ability of the test compound to interact with the receptor, wherein determining the ability of the test compound to interact with the receptor comprises determining the ability of the test compound to preferentially bind to the receptor as compared to the ability of the ligand, or a biologically active portion thereof, to bind to the receptor.

In another embodiment, an assay is a cell-based assay comprising contacting a cell expressing a particular target molecule described herein with a test compound and determining the ability of the test compound to modulate or alter (e.g. stimulate or inhibit) the activity of the target molecule. Determining the ability of the test compound to modulate the activity of the target molecule can be accomplished, for example, by determining the ability of a known ligand to bind to or interact with the target molecule.

Determining the ability of the known ligand to bind to or interact with the target molecule can be accomplished by one of the methods described above for determining direct binding. In a preferred embodiment, determining the ability of the known ligand to bind to or interact with the target molecule can be accomplished by determining the activity of the target molecule. For example, the activity of the target molecule can be determined by detecting induction of a cellular second messenger of the target (e.g., intracellular Ca.sup.2+, diacylglycerol, IP.sub.3, etc.), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a cellular response, for example, development, differentiation or rate of proliferation.

In yet another embodiment, an assay of the present invention is a cell-free assay in which protein of the invention or biologically active portion thereof is contacted with a test compound and the ability of the test compound to bind to the protein or biologically active portion thereof is determined. Binding of the test compound to the protein can be determined either directly or indirectly as described above. In one embodiment, the assay includes contacting the protein or biologically active portion thereof with a known compound which binds the protein to form an assay mixture, contacting the assay mixture with a test compound, and determining the ability of the test compound to interact with the protein. Determining the ability of the test compound to interact with the protein comprises determining the ability of the test compound to preferentially bind to the protein or biologically active portion thereof as compared to the known compound.

In another embodiment, the assay is a cell-free assay in which a protein of the invention or biologically active portion thereof is contacted with a test compound and the ability of the test compound to modulate or alter (e.g., stimulate or inhibit) the activity of the protein or biologically active portion thereof is determined. Determining the ability of the test compound to modulate the activity of the protein can be accomplished, for example, by determining the ability of the protein to bind to a known target molecule by one of the methods described above for determining direct binding. Determining the ability of the protein to bind to a target molecule can also be accomplished using a technology such as real-time Bimolecular Interaction Analysis (BIA). Sjolander, S. and Urbaniczky, C. (1991) Anal. Chem., 63:2338-2345 and Szabo et al. (1995) Curr. Opin. Struct. Biol., 5:699-705. As used herein, “BIA” is a technology for studying biospecific interactions in real time, without labeling any of the interactants (e.g., BIAcore.TM.) . Changes in the optical phenomenon surface plasmon resonance (SPR) can be used as an indication of real-time reactions between biological molecules.

In an alternative embodiment, determining the ability of the test compound to modulate the activity of a protein of the invention can be accomplished by determining the ability of the protein to further modulate the activity of a target molecule. For example, the catalytic/enzymatic activity of the target molecule on an appropriate substrate can be determined as previously described.

In yet another embodiment, the cell-free assay involves contacting a protein of the invention or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the protein to preferentially bind to or modulate the activity of a target molecule.

The cell-free assays of the present invention are amenable to use of both soluble and/or membrane-bound forms of isolated proteins. In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton.RTM.X-100, Triton.RTM.X-114, Thesit.RTM., Isotridecypoly(ethylene glycol ether). sub.n,3-[(3-cholamidopropyl)dimethylamminio]-1-propane sulfonate (CHAPS), 3-[(3-cholamidopropyl)dimethylamminio]2-hydroxy-1-propane sulfonate (CHAPSO), or N-dodecyl-N,Ndimethyl-3-ammonio-1-propane sulfonate.

In more than one embodiment of the above assay methods of the present invention, it may be desirable to immobilize either the protein or its target molecule to facilitate separation of complexed from uncomplexed forms of one or both of the proteins, as well as to accommodate automation of the assay. Binding of a test compound to the protein, or interaction of the protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or protein of the invention, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either a protein of the invention or a target molecule can be immobilized utilizing conjugation of biotin and streptavidin. Biotinylated protein of the invention or target molecules can be prepared from biotin-NHS(N-hydroxysuccinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the invention or target molecules, but which do not interfere with binding of the protein to its target molecule, can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays which rely on detecting an enzymatic activity associated with the protein or target molecule.

In another embodiment, modulators of expression of nucleic acid molecules of the invention are identified in a method wherein a cell is contacted with a candidate compound and the expression of appropriate mRNA or protein in the cell is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater (statistically significantly greater) in the presence of the candidate compound than in its absence, the candidate compound is identified as a stimulator or enhancer of the mRNA or protein expression. Alternatively, when expression of the mRNA or protein is less (statistically significantly less) in the presence of the candidate compound than in its absence, the candidate compound is identified as an inhibitor of the mRNA or protein expression. The level of rnRNA or protein expression in the cells can be determined by methods described herein for detecting mRNA or protein.

In yet another aspect of the invention, the proteins of the invention can be used as “bait proteins” in a two-hybrid assay or three-hybrid assay (see, e.g., U.S. Pat. No. 5,283,317; Zervos et al. (1993) Cell, 72:223-232; Madura et al. (1993) J. Biol. Chem., 268:12046-12054; Bartel et al. (1993) Biotechniques, 14:920-924; Iwabuchi et al. (1993) Oncogene, 8:1693-1696; and Brent W094/10300), to identify other proteins (captured proteins) which bind to or interact with the proteins of the invention and modulate their activity. Such captured proteins are also likely to be involved in the propagation of signals by the proteins of the invention as, for example, downstream elements of a protein-mediated signaling pathway. Alternatively, such captured proteins are likely to be cell-surface molecules associated with non-protein-expressing cells, wherein such captured proteins are involved in signal transduction.

The two-hybrid system is based on the modular nature of most transcription factors, which consist of separable DNA-binding and activation domains. Briefly, the assay utilizes two different DNA constructs. In one construct, the gene that codes for a protein of the invention is fused to a gene encoding the DNA binding domain of a known transcription factor (e.g., GAL-4) . In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected, and cell colonies containing the functional transcription factor can be isolated and used to obtain the cloned gene which encodes the protein which interacts with the protein of the invention.

This invention further pertains to novel agents identified by the above-described screening assays. Accordingly, it is within the scope of this invention to further use an agent identified as described herein in an appropriate animal model. For example, an agent identified as described herein (e.g., a modulating agent, an antisense nucleic acid molecule, a specific antibody, or a protein-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Animal models appropriate for the study of the progression of OA include the anterior cruciate ligament dog model (Rubin et al., Clin. Orthop. 113:212 (1975); Johnson, Exp. Pathol. 30:209 (1986); Pelletier et al., Osteoarthritis Cartilage 7:416 (1999)), the rabbit partial meniscectomy model (Colombo et al., Arthritis Rheum. 26:875 (1983); Butler et al., Arthritis Rheum. 26:1380 (1983)), and the guinea pig spontaneous model (Watson et al., Osteoarthritis Cartilage 4:197 (1996); Jiminez et al., Lab Anim. Sci. 47:598 (1997)).

4. Detection Assays

Portions or fragments of the nucleotide sequences identified herein (and the corresponding complete gene sequences) can be used as polynucleotide reagents. For example, the sequences can be used to map their respective genes on a chromosome and thus locate gene regions associated with genetic disease.

a. Chromosome Mapping

Once the sequence (or a portion of the sequence) of a gene has been isolated, this sequence can be used to map the location of the gene on a chromosome. This process is called chromosome mapping. Accordingly, portions or fragments of the nucleic acid molecules, described herein, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-30 bp in length) from the nucleic acid molecules described herein. Computer analysis of the sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the appropriate nucleotide sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science, 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the nucleic acid molecules of the invention to design oligonucleotide primers, sublocalization can be achieved with panels of fragments from specific chromosomes. Other mapping strategies which can similarly be used to map a specified sequence to its chromosome include in situ hybridization (described in Fan, Y et al. (1990) PNAS, 97:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. for a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Medelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J et al. (1987) Nature, 325:783-787.

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible form chromosome spreads or detectable using PCR based on that DNA sequence. Ultimately, complete sequencing of genes from several individuals can be performed to confirm the presence of a mutation and to distinguish mutations from polymorphisms.

5. Predictive Medicine

The present invention also pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining protein and/or nucleic acid expression as well as activity of proteins of the invention, in the context of a biological sample (e.g., blood, serum, cells, tissue) to thereby determine whether an individual is afflicted with a disease or disorder, or is at risk of developing a disorder, associated with aberrant expression or activity. The invention also provides for prognostic (or predictive) assays for determining whether an individual is at risk of developing a disorder associated with activity or expression of proteins or nucleic acids of the invention.

For example, mutations in a specified gene can be assayed in a biological sample. Such assays can be used for prognostic or predictive purpose to thereby phophylactically treat an individual prior to the onset of a disorder characterized by or associated with expression or activity of nucleic acid molecules or proteins of the invention.

Another aspect of the invention pertains to monitoring the influence of agents (e.g., drugs, compounds) on the expression or activity of proteins of the invention in clinical trials.

These and other agents are described in further detail in the following sections.

a. Diagnostic Assays

An exemplary method for detecting the presence or absence of proteins or nucleic acids of the invention in a biological sample involves obtaining a biological sample from a test subject and contacting the biological sample with a compound or an agent capable of detecting the protein, or nucleic acid (e.g., mRNA, genomic DNA) that encodes the protein, such that the presence of the protein or nucleic acid is detected in the biological sample. A preferred agent for detecting mRNA or genomic DNA is a labeled nucleic acid probe capable of hybridizing to mRNA or genomic DNA sequences described herein. The nucleic acid probe can be, for example, a full-length nucleic acid, or a portion thereof, such as an oligonucleotide of at least 15, 30, 50, 100, 250 or 500 nucleotides in length and sufficient to specifically hybridize under stringent conditions to appropriate mRNA or genomic DNA. Other suitable probes for use in the diagnostic assays of the invention are described herein.

A preferred agent for detecting proteins of the invention is an antibody capable of binding to the protein, preferably an antibody with a detectable label. Antibodies can be polyclonal, or more preferably, monoclonal. An intact antibody, or a fragment thereof (e.g., Fab or F(ab.sup.1) .sub.2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA of the invention in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled anti-protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample or biopsy isolated by conventional means from a subject.

In another embodiment, the methods further involve obtaining a control biological sample from a control subject, contacting the control sample with a compound or agent capable of detecting protein, mRNA, or genomic DNA of the invention, such that the presence of protein, mRNA or genomic DNA is detected in the biological sample, and comparing the presence of protein, mRNA or genomic DNA in the control sample with the presence of protein, mRNA or genomic DNA in the test sample.

The invention also encompasses kits for detecting the presence of proteins or nucleic acid molecules of the invention in a biological sample. For example, the kit can comprise a labeled compound or agent capable of detecting protein or mRNA in a biological sample; means for determining the amount of in the sample; and means for comparing the amount of in the sample with a standard. The compound or agent can be packaged in a suitable container. The kit can further comprise instructions for using the kit to detect protein or nucleic acid.

b. Prognostic Assays

The diagnostic methods described herein can furthermore be utilized to identify subjects having or at risk of developing a disease or disorder associated with aberrant expression or activity of proteins and nucleic acid molecules of the invention. For example, the assays described herein, such as the preceding diagnostic assays or the following assays can be utilized to identify a subject having or at risk of developing a disorder associated with protein or nucleic acid expression or activity such as OA. Alternatively, the prognostic assays can be utilized to identify a subject having or at risk for developing OA. Thus, the present invention provides a method for identifying a disease or disorder associated with aberrant expression or activity of proteins or nucleic acid molecules of the invention, in which a test sample is obtained from a subject and protein or nucleic acid (e.g., mRNA, genomic DNA) is detected, wherein the presence of protein or nucleic acid is diagnostic for a subject having or at risk of developing a disease or disorder associated with aberrant expression or activity of the protein or nucleic acid sequence of the invention. As used herein, a “test sample” refers to a biological sample obtained from a subject of interest. For example, a test sample can be a biological fluid (e.g., synovial fluid), cell sample, or tissue (e.g., cartilage tissue).

Furthermore, the prognostic assays described herein can be used to determine whether a subject can be administered an agent (e.g., an agonist, antagonist, peptidomimetic, protein, polypeptide, nucleic acid, small molecule, or other drug candidate) to treat a disease or disorder associated with aberrant expression or activity of a protein or nucleic acid molecule of the invention. For example, such methods can be used to determine whether a subject can be effectively treated with an agent for a disorder, such as OA. Alternatively, such methods can be used to determine whether a subject can be effectively treated with an agent for OA. Thus, the present invention provides methods for determining whether a subject can be effectively treated with an agent for a disorder associated with aberrant expression or activity of a protein or nucleic acid of the present invention, in which a test sample is obtained and protein or nucleic acid expression or activity is detected (e.g., wherein the abundance of particular protein or nucleic acid expression or activity is diagnostic for a subject that can be administered the agent to treat a disorder associated with aberrant expression or activity.)

The methods of the invention can also be used to detect genetic alterations in genes or nucleic acid molecules of the present invention, thereby determining if a subject with the altered gene is at risk for OA. In one embodiment, the method includes detecting, in a sample of cells from the subject, the presence or absence of a genetic alteration characterized by at least one of an alteration affecting the integrity of a gene encoding a particular protein, or the mis-expression of the gene. For example, such genetic alterations can be detected by ascertaining the existence of at least one of (1) a deletion of one or more nucleotides; (2) an addition of one or more nucleotides; (3) a substitution of one or more nucleotides, (4) a chromosomal rearrangement; (5) an alteration in the level of a messenger RNA transcript; (6) aberrant modification, such as of the methylation pattern of the genomic DNA; (7) the presence of a non-wild type splicing pattern of a messenger RNA transcript; (8) a non-wild type level; (9) allelic loss; and (10) inappropriate post-translational modification. As described herein, there are a large number of assay techniques known in the art which can be used for detecting alterations in a particular gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such an anchor PCR or RACE PCR, or, alternatively, in a ligation chain reaction (LCR) (see, e.g., Landegran et al. (1988) Science, 241:1077-1080; and Nakazawa et al. (1994) PNAS, 91:360-364), the latter of which can be particularly useful for detecting point mutations (see Abravaya et al. (1995) Nucleic Acids Res., 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA, 87:1874-1878), transcriptional amplification system (Kwoh, D. Y. et al., (1989) Proc. Natl. Acad. Sci. USA, 86:1173-1177), Q-Beta Replicase (Lizardi, P. M. et al., (1988) Bio/Technology, 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a given gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for sample, U.S. Pat. No. 5,498,531) ca be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotide probes (Cronin, M. T. et al. (1996) Human Mutation, 7:244-255; Kozal, M. J. et al. (1996) Nature Medicine, 2:753-759). For example, genetic mutations can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the gene and detect mutations by comparing the sequence of the gene from the sample with the corresponding wild-type (control) gene sequence. Examples of sequencing reactions include those based on techniques developed by Maxim and Gilbert ((1997) PNAS, 74:560) or Sanger ((1977) PNAS, 74:5463). It is also contemplated that any of a variety of automated sequencing procedures can be utilized when performing the diagnostic assays ((1995) Biotechniques, 19:448), including sequencing by mass spectrometry (see, e.g., PCT International Publication No. WO 94/16101; Cohen et al. (1996) Adv. Chromatogr., 36:127-162; and Griffin et al. (1993) Appl. Biochem. Biotechnol., 38:147-159).

Other methods for detecting mutations include methods in which protection from cleavage agents is used to detect mismatched bases in RNA/RNA or RNA/DNA heteroduplexes (Myers et al. (1985) Science, 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-standard duplexes are treated with an agent which cleaves single-stranded regions of the duplex such as which will exist due to base pair mismatches between the control and sample strands. For instance, RNA/DNA duplexes can be treated with Rnase and DNA/DNA hybrids treated with S1 nuclease to enzymatically digest the mismatched regions. After digestion of the mismatched regions, the resulting material is then separated by size on denaturing polyacrylamide gels to determine the site of mutation. See, for example Cotton et al. (1988) Proc. Natl. Acad. Sci. USA, 85:4397; Saleeba et al. (1992) Methods Enzymol., 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair”, enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis, 15:1657-1662). According to an exemplary embodiment, a probe based on an nucleotide sequence of the invention is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA, 86:2766, see also Cotton (1993) Mutat Res, 285:125-144; and Hayashi (1992) Genet Anal. Tech. Appl., 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet., 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature, 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem., 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature, 324:163); Saiki et al. (1989) Proc. Natl. Acad. Sci. USA, 86:6320). Such allele-specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res., 17:2437-2448) or at the extreme 3′ end of one primer where, under appropriate conditions, mismatch can prevent, or reduce polymerase extension (Prossner (1993) Tibtech, 11:238). In addition it may be desirable to introduce a novel restriction site in the region of the mutation to create cleavage-based detection (Gasparini et al. (1992) Mol. Cell Probes, 6:1). It is anticipated that in certain embodiments amplification may also be performed using Taq ligase for amplification (Barany (1991) Proc. Natl. Acad. Sci. USA, 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene of the present invention. Any cell type or tissue in which the gene is expressed may be utilized in the prognostic assays described herein.

c. Monitoring of Effects During Clinical Trials

Monitoring the influence of agents (e.g., drugs, compounds) on the expression or activity of nucleic acid molecules or proteins of the present invention (e.g., modulation of cellular signal transduction, regulation of gene transcription in a cell involved in development or differentiation, regulation of cellular proliferation) can be applied not only in basic drug screening, but also in clinical trials. For example, the effectiveness of an agent determined by a screening assay as described herein to increase gene expression, protein levels, or upregulate protein activity, can be monitored in clinical trails of subjects exhibiting decreased gene expression, protein levels, or downregulated protein activity. Alternatively, the effectiveness of an agent determined by a screening assay to decrease gene expression, protein levels, or downregulate protein activity, can be monitored in clinical trials of subjects exhibiting increased gene expression, protein levels, or upregulated protein activity. In such clinical trials, the expression or activity of the specified gene and, preferably, other genes that have been implicated in, for example, a proliferative disorder can be used as a “read out” or markers of the phenotype of a particular cell.

For example, and not by way of limitation, genes that are modulated in cells by treatment with an agent (e.g., compound, drug or small molecule) which modulates protein activity (e.g., identified in a screening assay as described herein) can be identified. Thus, to study the effect of agents on OA, for example, in a clinical trial, cells can be isolated and RNA prepared and analyzed for the levels of expression of the specified gene and other genes implicated in OA. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of the specified gene or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, polypeptide, nucleic acid, small molecule, or other drug candidate identified by the screening assays described herein) comprising the steps of (I) obtaining a pre-administration sample from a subject prior to administration of the agent; (ii) detecting the level of expression of a specified protein, mRNA, or genomic DNA of the invention in the pre-administration sample; (iii) obtaining one or more post-administration samples from the subject; (iv) detecting the level of expression or activity of the protein, mRNA, or genomic DNA in the post-administration samples; (v) comparing the level of expression or activity of the protein, mRNA, or genomic DNA in the pre-administration sample with the protein, mRNA, or genomic DNA in the post-administration sample or samples; and (vi) altering the administration of the agent to the subject accordingly. For example, increased administration of the agent may be desirable to increase the expression or activity of the protein or nucleic acid molecule to higher levels than detected, i.e., to increase effectiveness of the agent. Alternatively, decreased administration of the agent may be desirable to decrease effectiveness of the agent. According to such an embodiment, protein or nucleic acid expression or activity may be used as an indicator of the effectiveness of an agent, even in the absence of an observable phenotypic response.

6. Methods of Treatment

The present invention provides for both prophylactic and therapeutic methods of treating a subject at risk of (or susceptible to) a disorder or having a disorder associated with aberrant expression or activity of proteins or nucleic acids of the invention. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with the molecules of the present invention or modulators according to that individual's drug response genotype. Pharmacogenomics allows a clinician or physician to target prophylactic or therapeutic treatments to patients who will most benefit from the treatment and to avoid treatment of patients who will experience toxic drug related side effects.

a. Prophylactic Methods

In one aspect, the invention provides a method for preventing in a subject, a disease or condition associated with aberrant expression or activity of genes or proteins of the present invention, by administering to the subject an agent which modulates expression or at least one activity of a gene or protein of the invention. Subjects at risk for a disease which is caused or contributed to by aberrant gene expression or protein activity can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein. The prophylactic methods of the present invention are further discussed in the following subsections.

b. Therapeutic Methods

Another aspect of the invention pertains to methods of modulating expression or activity of genes or proteins of the invention for therapeutic purposes. The modulatory method of the invention involves contacting a cell with an agent that modulates one or more of the activities of the specified protein associated with the cell. An agent that modulates protein activity can be an agent as described herein, such as a nucleic acid or a protein, a naturally-occurring target molecule of a protein described herein, a polypeptide, a peptidomimetic, or other small molecule. In one embodiment, the agent stimulates one or more protein activities. Examples of such stimulatory agents include active protein as well as a nucleic acid molecule encoding the protein that has been introduced into the cell. In another embodiment, the agent inhibits one or more protein activities. Examples of such inhibitory agents include antisense nucleic acid molecules and anti-protein antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a protein or nucleic acid molecule of the invention. In one embodiment, the method involves administering an agent (e.g., an agent identified by a screening assay described herein), or combination of agents that modulates (e.g., upregulates or downregulates) expression or activity of a gene or protein of the invention. In another embodiment, the method involves administering a protein or nucleic acid molecule of the invention as therapy to compensate for reduced or aberrant expression or activity of the protein or nucleic acid molecule.

Stimulation of protein activity is desirable in situations in which the protein is abnormally downregulated and/or in which increased protein activity is likely to have a beneficial effect. Likewise, inhibition of protein activity is desirable in situations in which the protein is abnormally upregulated and/or in which decreased protein activity is likely to have a beneficial effect.

c. Pharmacogenomics

The molecules of the present invention, as well as agents, or modulators which have a stimulatory or inhibitory effect on the protein activity (e.g., gene expression) as identified by a screening assay described herein can be administered to individuals to treat (prophylactically or therapeutically) disorders (e.g., proliferative or developmental disorders) associated with aberrant protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a molecule of the invention or modulator thereof, as well as tailoring the dosage and/or therapeutic regimen of treatment with such a molecule or modulator.

Pharmacogenomics deals with clinically significant hereditary variations in the response to drugs due to altered drug disposition and abnormal action in affected persons. See e.g., Eichelbaum, M., Clin Exp Pharmacol. Physiol., (1996) 23(10-11): 983-985 and Linder, M. W., Clin. Chem. (1997) 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1,000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a protein or a receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2(NAT 2) and cytochrme P450 enzymes CYP2D6 and CYP2C19) has provided an explanation as to why some patients do not obtain the expected drug effects or show exaggerated drug response and serious toxicity afer taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a molecule or modulator of the present invention) can given an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a molecule or modulator of the invention, such as a modulator identified by one of the exemplary screening assays described herein.

The contents of all references, patents and published patent applications cited throughout this application are hereby incorporated by reference.

### EXAMPLES

The following examples will illustrate the invention in greater detail, although it will be understood that the invention is not limited to these specific examples. Various other examples will be apparent to the person skilled in the art after reading the present disclosure without departing from the spirit and scope of the invention. It is intended that all such other examples be included within the scope of the appended claims.

### Example 1

Identification of Initial ESTs

Highly selective targets for osteoarthritic tissue were identified from ostetoarthritic tissue to a global database of ESTs selected from libraries made from normal human tissue. Three EST libraries of OA cartilage and synovium from 6 different donors (five cartilage donors and one synovium donor) were compared to two EST libraries representing cartilage and synovium from 5 donors (four cartilage donors and one synovium donor) without OA to identify those ESTs which are representative of genes upregulated as a result of OA. The data mining effort used sequence comparison techniques based on BLAST comparison of individual ESTs to evaluate which ESTs were preferentially observed in the target libraries versus control and/or normal libraries. The resulting EST sequences represent osteoarthrits tissue-derived nucleic acids of the invention and can be used to create additional osteoarthrits tissue-derived nucleic acids, proteins, and polypeptides of the invention. The initial ESTs identified from osteoarthrits tissue libraries (Table 1) were run through bioinformatic computer analysis for the presence of specific structural features, and were given the best possible annotation. Table 1 correlates that information. The sequences which were found to be secreted proteins seen at a frequency which statistically correlates with OA disease presence appear in Table 2.

Table 1. Nucleic acid molecules expressed in osteoarthrits tissue with potential uses for the development of human therapeutics and diagnostics.

Table 2: Preferred candidate markers of OA Example 2

### Example 2

Strategy for obtaining FL clones

The sequences, or fragments of them, disclosed can be used to directly obtain full length clones from cDNA libraries and genomic clones from genomic libraries. A number of methods have been described to obtain cDNA and genomic clones, including 5′ RACE (for example, using the Marathon cDNA Amplification Kit from Clonetech, Inc.), Genetrapper (LifeTechnologies, Inc.), colony hybridization (Sambrook et al., 1989, Molecular Cloning, A Laboratory Manual 2nd Ed., Cold Spring Harbor Press, Cold Spring Harbor, N.Y.) and array hybridization. One skilled in the art can refer to general reference texts for detailed descriptions of known techniques or equivalent techniques. These texts include Current Protocols in Molecular Biology (Ausubel, et al., eds., John Wiley & Sons, N.Y. (1989), and supplements through September 1998), Molecular Cloning, A Laboratory Manual (Sambrook et al., 2nd Ed., Cold Spring Harbor Press, Cold Spring Harbor, N.Y. (1989)), and Current Protocols in Immunology (Coligan, ed., John Wiley and Sons, Toronto (1994)), for example, each of which are specifically incorporated by reference in their entirety.

A preferred method is array hybridization. A first step in array hybridization is to obtain a high quality library with high complexity and a high proportion of full length or high molecular weight clones. A cDNA library can be purchased from a number of commercial sources or a new library prepared from mRNA derived from tissue known or suspected to express the gene. Details on library construction can be found in Sambrook et al. (Molecular Cloning, A Laboratory Manual 2nd Ed., Cold Spring Harbor Press, Cold Spring Harbor, N.Y.). Similarly, genomic libraries may be purchased from commercial sources.

A plasmid cDNA library constructed from human OA tissue -derived mRNA is plated on agar and a picking robot (for example, a ‘Q’ BOT from Genetix, Inc.) is used to pick individual colonies into 96 well plates containing LB medium. The isolated E. coli cells are grown overnight at 30° C. and placed at 4° C. until ready for spotting. The E. coli are then robotically spotted in high density grids on nitrocellulose membranes overlaying solid agar medium. Preferably, each colony is double spotted at two different locations. The colonies are allowed to grow to 0.1-0.2 cm and then the membranes are prepared for hybridization to nucleic acid sequences. Standard protocols for membrane preparation can be found in Sambrook (Molecular Cloning, A Laboratory Manual 2nd Ed., Cold Spring Harbor Press, Cold Spring Harbor, N.Y.).

Selection of nucleic acid probes, or similarly PCR primers, can be done using a variety of methods, including publicly available programs such as MIT Primer 3 (Rozen and Skaletsky (1996, 1997, 1998)), for example.

Synthetic oligos or a restriction fragment containing probe sequences, from the original clone sequence, are labeled with ³³P using conventional means, such as the random hexamer priming method (High Prime kit from Boehringer Mannheim). The labeled probes are then hybridized to the arrayed filters under stringent conditions (Sambrook et al., 1989, Molecular Cloning, A Laboratory Manual 2nd Ed., Cold Spring Harbor Press, Cold Spring Harbor, N.Y.). In this case, the wash conditions can approach 70-80° C. at 0.1M NaCl. After washing, the positive colonies can be visualized by exposing the blots to film or to a phosphoimaging screen. The processed image reveals colonies containing cDNA clones of the targeted gene. Alternatively, a non-radioactive method might be chosen to label the probes and detect colony hybridization (for example, the DIG Non-Radioactive DNA Labeling and Detection Kit from Boehringer Mannheim). Each clone is analyzed by restriction digests to identify the longest clone in the group. The longer clones are sequenced using an automated sequencing system (for example a Perkin-Elmer ABI 377) and the sequences evaluated for a complete open reading frame initiated with a methionine start codon (ATG). Genomic clones can also be analyzed for intron-exon boundaries using known methods as well as compared to relevant or homologous gene or protein sequence information to determine the coding regions.

### Example 3

Expression and Purification of a Polypeptide in E. coli Host

The vector pProEX HT is used for expression of a polypepetide in a bacterial host system. (LifeTechnologies Inc., Gaithersburg, MD). The plasmid encodes the ampicillin resistance gene (“Apr”) and contains a pBR322 origin of replication (ori), an IPTG inducible promoter, a ribosome binding site, and six codons encoding a histidine tag at the amino terminus. The his tag allows affinity purification using immobilized metal ion affininty chromatography (IMAC), such as with the nickel-nitrilo-tri-acetic acid (Ni-NTA) affinity resin. The cloning region contains suitable restriction enzyme cleavage sites for insertion of polypeptide encoding sequences. The vector also encodes a TEV (Tobbacco Etch Virus) protease cleavage site to remove the his tag region from the amino terminus of the expressed polypeptide.

The desired polypeptide encoding sequence or fragment, typically lacking any hydrophobic leader sequence, is PCR amplified from a cDNA clone. Primers designed from at least one of SEQ NO:1-82, which anneal to the amino terminal sequences of the desired polypeptide encoding region, and from the 3′ end of the cDNA clone, preferably within or at the beginning of vector sequences of the cDNA clone, are used. Additional sequence containing restriction sites, to facilitate cloning into pProEX HT vector, or stop codons can be incorporated into one or both of the primers.

PCR amplified sequences and the vector are digested with appropriate restriction enzymes and then ligated together. Insertion of the DNA into the digested pProEX HT vector places a polypeptide encoding sequence downstream from the trc promoter and in proper reading frame to an initiator AUG.

The ligation mixture is transformed into competent E. coli cells using standard procedures. Sambrook, et al., 1989; Ausubel, 1989, supplements through September 1998. E. coli strain DH5a is used, however other strains are possible. Amp resistant colonies indicate successful transformation. Plasmid DNA from resistant colonies is isolated and the correct construction confirmed by restriction analysis, PCR, and DNA sequencing.

Clones containing the desired constructs are grown over night in LB media supplemented with amp (100 mg/ml). These culture are used to inoculate production cultures, where the cells are grown at 370C to an OD590 density of between about 0.4 and 0.6 before induction by adding isopropyl-b-D-thiogalactopyranoside (IPTG) to a final concentration of 1 mM, and allowed an additional 3 to 4 hours to express polypeptide. Cells then are harvested by centrifugation.

The cell pellet is then brought up in 6M guanidine-HCl, pH8, at 4° C., and stirred for 4 hours. The cell debris is removed by centrifugation and the supernatant containing the expressed polypeptide is dialyzed against a refolding buffer, such as a NaCl-based or Tris-based buffer, at about pH 6 and containing protease inhibitors. After refolding, the polypeptide is purified by IMAC and optionally cleaved with TEV protease. The cleavage reaction is run through a size exclusion gel to purify the desired polypeptide. Purified polypeptide is stored at 4° C. or frozen at −80° C. Gel electrophoresis can be used to verify production of desired polypeptide or for purification.

As noted above, the specific examples should not be interpreted as a limitation to the scope of the invention. Instead, they are merely exemplary embodiments one skilled in the art would understand from the entire disclosure of this invention.

TABLE 1*

| Seq NO | Hit Description | P Score |
|---|---|---|
| SEQ NO 1 | Human mRNA for fibronectin (FN precursor | 100 |
| SEQ NO 2 | Human lumican mRNA, complete cds. | 100 |
| SEQ NO 3 | Human ferritin H chain mRNA, complete cd | 100 |
| SEQ NO 4 | Human osteoprotegerin (OPG) mRNA, comple | 100 |
| SEQ NO 5 | Human DNA for osteopontin, complete cds. | 100 |
| SEQ NO 6 | Human complement decay-accelerating fact | 100 |
| SEQ NO 7 | Human SOD-2 gene for manganese superoxid | 100 |
| SEQ NO 8 | Human chitinase (HUMTCHIT) mRNA, exon 1b | 100 |
| SEQ NO 9 | Human CRTL1 gene exons 1, 2, 3, 4 and 5. | 100 |
| SEQ NO 10 | Human glia-derived nexin (GDN) mRNA, 5′ | 100 |
| SEQ NO 11 | Human SPARC/osteonectin mRNA, complete c | 100 |
| SEQ NO 12 | Human mRNA for CD59, an LY-6-like protei | 100 |
| SEQ NO 13 | Human mRNA for pro-alpha-1 type 3 collag | 100 |
| SEQ NO 14 | Human mRNA for eukaryotic initiation fac | 100 |
| SEQ NO 15 | Human mRNA for DIF-2 protein. | 100 |
| SEQ NO 16 | Human apolipoprotein J mRNA, complete cd | 100 |
| SEQ NO 17 | Human chondroitin/dermatan sulfate prote | 100 |
| SEQ NO 18 | Human alpha-1-antichymotrypsin mRNA, 3′ | 100 |
| SEQ NO 19 | Human alpha-1-antitrypsin mRNA, complete | 100 |
| SEQ NO 20 | Human mRNA for serine protease. | 100 |
| SEQ NO 21 | Human mRNA for caveolin. | 96 |
| SEQ NO 22 | Human G0S2 protein gene, complete cds. | 100 |
| SEQ NO 23 | Human mRNA for CC chemokine LARC precurs | 100 |
| SEQ NO 24 | Human mRNA for proliferation-associated | 100 |
| SEQ NO 25 | Human mRNA for collagen VI alpha-2 C-ter | 100 |
| SEQ NO 26 | Human fos proto-oncogene (c-fos), comple | 100 |
| SEQ NO 27 | Human zinc finger transcriptional regula | 100 |
| SEQ NO 28 | Human zinc finger protein (MAZ) mRNA. | 100 |
| SEQ NO 29 | Human muscle creatine kinase gene (CKMM) | 100 |
| SEQ NO 30 | FBLN1; fibulin-1, isoform D precursor | 9 |
| SEQ NO 31 | Human skeletal muscle LIM-protein FHL1 m | 100 |
| SEQ NO 32 | Human caldesmon mRNA, complete cds. | 100 |
| SEQ NO 33 | Human slow skeletal muscle troponin T mR | 100 |
| SEQ NO 34 | Human mRNA for nuclear envelope protein | 100 |
| SEQ NO 35 | Human mRNA for titin protein (clone hhl- | 100 |
| SEQ NO 36 | Human alpha-B-crystallin gene, 5′ end. | 100 |
| SEQ NO 37 | Human suilisol mRNA, complete cds. | 100 |
| SEQ NO 38 | Human CAPL protein mRNA, complete cds. | 100 |
| SEQ NO 39 | Human mRNA for KIAA0253 gene, partial cd | 100 |
| SEQ NO 40 | Human tenascin-X (XA) mRNA, complete cds | 100 |
| SEQ NO 41 | Human gene for fibromodulin. | 100 |
| SEQ NO 42 | Human RASF-A PLA2 mRNA, complete cds. | 100 |
| SEQ NO 43 | Human mRNA for activin beta-A, exon1,2. | 100 |
| SEQ NO 44 | Human tumor necrosis factor-inducible (T | 100 |
| SEQ NO 45 | nucleotide pyrophosphohydrolase | 100 |
| SEQ NO 46 | Human Pig12 (PIG12) mRNA, complete cds. | 100 |
| SEQ NO 47 | Human mRNA for cysteine dioxygenase, com | 100 |
| SEQ NO 48 | Human sialoprotein mRNA, complete cds. | 100 |
| SEQ NO 49 | Human mRNA for alpha-1 type II collagen. | 100 |
| SEQ NO 50 | Human alpha-2 type XI collagen mRNA (COL | 100 |
| SEQ NO 51 | F08D12.3 | 4 |
| SEQ NO 52 | Rat chondroadherin mRNA, complete cds. | 76 |
| SEQ NO 53 | ChM-I; chondromodulin-I | 76 |
| SEQ NO 54 | mimecan | 95 |
| SEQ NO 55 | Human chondroadherin gene, 5′flanking re | 100 |
| SEQ NO 56 | Human bone morphogenetic protein 2A (BMP | 30 |
| SEQ NO 57 | Human (lambda) DNA for Ig light chain. | 17 |
| SEQ NO 58 | | 0 |
| SEQ NO 59 | | 0 |
| SEQ NO 60 | | 0 |
| SEQ NO 61 | | 0 |
| SEQ NO 62 | | 0 |
| SEQ NO 63 | | 0 |
| SEQ NO 64 | | 0 |
| SEQ NO 65 | | 0 |
| SEQ NO 66 | | 0 |
| SEQ NO 67 | | 0 |
| SEQ NO 68 | | 0 |
| SEQ NO 69 | | 0 |
| SEQ NO 70 | | 0 |
| SEQ NO 71 | | 0 |
| SEQ NO 72 | | 0 |
| SEQ NO 73 | | 0 |
| SEQ NO 74 | | 0 |
| SEQ NO 75 | | 0 |
| SEQ NO 76 | | 0 |
| SEQ NO 77 | Human mRNA for scrapie responsive protein | 78 |
| SEQ NO 78 | | 0 |
| SEQ NO 79 | | 0 |
| SEQ NO 80 | | 0 |
| SEQ NO 81 | | 0 |
| SEQ NO 82 | | 0 |

TABLE 2

| Seq NO | Hit Description | P Score |
|---|---|---|
| SEQ NO 1 | Human mRNA for fibronectin (FN precursor | 100 |
| SEQ NO 2 | Human lumican mRNA, complete cds. | 100 |
| SEQ NO 3 | Human ferritin H chain mRNA, complete cd | 100 |
| SEQ NO 4 | Human osteoprotegerin (OPG) mRNA, comple | 100 |
| SEQ NO 5 | Human DNA for osteopontin, complete cds. | 100 |
| SEQ NO 6 | Human complement decay-accelerating fact | 100 |
| SEQ NO 8 | Human chitinase (HUMTCHIT) mRNA, exon 1b | 100 |
| SEQ NO 9 | Human CRTL1 gene exons 1, 2, 3, 4 and 5. | 100 |
| SEQ NO 10 | Human glia-derived nexin (GDN) mRNA, 5′ | 100 |
| SEQ NO 11 | Human SPARC/osteonectin mRNA, complete c | 100 |
| SEQ NO 12 | Human mRNA for CD59, an LY-6-like protei | 100 |
| SEQ NO 30 | FBLN1; fibulin-1, isoform D precursor | 9 |

We claim:

1. A nucleic acid having a nucleotide sequence selected from the group consisting of SEQ NO: 1 through SEQ NO: 82, or complements thereof.

2. The nucleic acid capable of specifically hybridizing to a nucleic acid of claim 1, or a complement thereof.

3. The nucleic acid exhibiting a percentage identity of between about 70% to about 90% with at least a 10 nucleotide region of the sequence of a nucleic acid of claim 2.

4. The nucleic acid exhibiting a percentage identity of between about 90% to about 99% with at least a 10 nucleotide region of the sequence of a nucleic acid of claim 3.

5. The nucleic acid as claimed in any one of claims 3 or 4 wherein said nucleic acid is detectably labeled.

6. The nucleic acid of claim 5 wherein said sequence is a marker of osteoarthritis progression.

7. The nucleic acid of claim 5 wherein said label is selected from the group consisting of radioactive, fluorescent, chemi-luminescent, and chromogenic agents, and magnetic particles.

8. A method of identifying a nucleic acid comprising contacting a hybridization probe as claimed in claim 5 with a sample containing nucleic acid and detecting hybridization to the hybridization probe.

9. A method of identifying a nucleic acid comprising contacting a PCR probe as claimed in claim 5 with a sample containing nucleic acid and producing multiple copies of a nucleic acid that hybridizes to the PCR probe.

10. A substantially-purified nucleic acid having at least one 10 nucleotide region substantially identical to a sequence identified in Table 1.

11. A recombinant DNA comprising a nucleic acid according to one of claims 1-4, or 8, wherein the recombinant nucleic acid further comprises a promoter or partial promoter region.

12. A host cell containing a nucleic acid as claimed in claim 10.

13. A method for producing and purifying a polypeptide, said method comprising the steps of:
a) culturing the host cell of claim 12 under conditions suitable for the expression of the peptide; and
b) recovering the polypeptide from the host cell culture.

14. A substantially-purified protein, polypeptide, or fragment thereof, wherein at least one 15 amino acid region is encoded by a nucleic acid as claimed in one of claims 1-4.

15. An antibody that specifically binds to a purified protein, polypeptide, or fragment thereof, having at least one region of 5 contiguous amino acids encoded by a nucleic acid as claimed in one of claims 1-4 or 10.

16. A transgenic animal having in one or more of its cells an introduced nucleic acid as claimed in one of claims 1-4 or 10, or progeny of the transgenic animal.

17. A cell taken from a transgenic animal or its progeny as claimed in claim 16.

18. A composition comprising a nucleic acid as claimed in one of claims 1-3, or a complement thereof.

19. A method of identifying a biologically active compound or composition comprising contacting the compound or composition with a sample comprising a protein, polypeptide, or fragment as claimed in 13, and comparing the interaction between the compound or composition and the protein, polypeptide, or fragment with a control.

20. A compound or composition that is detectable in a method of claim 18.

21. A computer-readable medium having recorded thereon the sequence information of one or more of SEQ NO: 1 through SEQ NO: 82 or complements thereof.

22. A method of identifying a nucleic acid comprising providing a computer-readable medium as claimed in claim 21 and comparing nucleotide sequence information using a computerized means.

23. A substantially-purified nucleic acid molecule which comprises a nucleic acid sequence that is identical to at least 10 nucleotides of a nucleotide sequence selected from the group consisting of SEQ NO: 1 through SEQ NO: 82, or complements thereof.

24. A substantially-purified nucleic acid molecule which comprises a nucleic acid sequence that is identical to at least 50 nucleotides of a nucleotide sequence selected from the group consisting of SEQ NO: 1 through SEQ NO: 82, or complements thereof.

25. A substantially-purified nucleic acid molecule which comprises a nucleic acid sequence that is identical to at least 100 nucleotides of a nucleotide sequence selected from the group consisting of SEQ NO: 1 through SEQ NO: 82, or complements thereof.

26. A substantially-purified protein, polypeptide, or fragment thereof, of claim 13 wherein said substantially-purified protein, polypeptide, or fragment thereof is a fusion protein.

27. An antibody of claim that is detectably-labeled.

28. A transformed cell having a nucleic acid molecule of claim 1.

28. A transformed cell having the antisense of a nucleic acid molecule of claim 1.

29. A process for diagnosis or prognosis of osteoarthritis a mammal from the expression of mRNA or cDNA that is identical to at least 20 nucleotides of a nucleotide sequence selected from the group consisting of SEQ NO: 1 through SEQ NO: 82, or complements thereof.

30. A method of isolating a nucleic acid that is identical to at least 20 nucleotides of a nucleotide sequence selected from the group consisting of SEQ NO: 1 through SEQ NO: 82, or complements thereof.
